(19) United States
(12) Patent Application Publication
(10) Pub. No.: US 2016/0362437 A1
(43) Pub. Date: Dec. 15, 2016

(54) Method for Producing Lentztrehalose A, Compound Useful for the Method, and Method for Producing the Compound

(71) Applicant: Microbial Chemistry Research Foundation, Tokyo (JP)

(72) Inventors: Takumi WATANABE, Tokyo (JP); Shunichi WADA, Tokyo (JP); Masayuki IGARASHI, Tokyo (JP); Ming ZHANG, Tokyo (JP)

(21) Appl. No.: 15/170,058

(22) Filed: Jun. 1, 2016

(30) Foreign Application Priority Data
Jun. 11, 2015 (JP) 2015-118218
Jul. 30, 2015 (JP) 2015-150626

(51) Int. Cl. C07H 15/04; C07H 1/00; C07H 15/203

(52) U.S. Cl. CPC C07H 15/04; C07H 15/203; C07H 1/00

(57) ABSTRACT

A method for producing a compound represented by Structural Formula (1), including: introducing benzyl group into trehalose to produce at least one of compound represented by Structural Formula 4a and compound represented by Structural Formula 4a′; subjecting at least one of the Structural Formula 4a compound and the Structural Formula 4a′ compound to prenylation to produce at least one of compound represented by Structural Formula 3a and compound represented by Structural Formula 3a′; subjecting at least one of the Structural Formula 3a compound and the Structural Formula 3a′ compound to sharpless asymmetric dihydroxylation to produce at least one of compound represented by Structural Formula 2a and compound represented by Structural Formula 2a′; and allowing at least one of the Structural Formula 2a compound and the Structural Formula 2a′ compound to react with hydrogen in the presence of palladium catalyst to produce the compound represented by Structural Formula (1).

### BACKGROUND OF THE INVENTION

Field of the Invention

The present invention relates to a method for producing a compound represented by the following Structural Formula (1) (hereinafter may be referred to as “lentztrehalose A”), a compound useful for the method for producing lentztrehalose A, and a method for producing the compound useful for the method for producing lentztrehalose A:

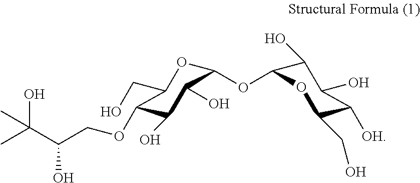

Description of the Related Art

Lentztrehalose A is a very useful compound from the viewpoints of its excellent anti-tumor effect, bone reinforcing effect, or weight-gain suppressing effect, and its availability as a sweeting agent (see, Japanese Patent Application Laid-Open (JP-A) No. 2014-227404 and Wada S et al, Structure and biological properties of lentztrehalose: a novel trehalose analog, J Antibiot (Tokyo). 2014 April; 67 (4): 319-22).

The lentztrehalose A has been conventionally produced through bacterial biosynthesis, but satisfactory yield has not been achieved. Therefore, there is a need for an improved synthetic method.

### SUMMARY OF THE INVENTION

The present invention aims to solve the above existing problems and achieve the following object. That is, an object of the present invention is to provide a method for producing lentztrehalose A with high efficiency, an intermediate useful for producing the lentztrehalose A, and a method for producing the intermediate.

Means for solving the above problem are as follows.

<1> A method for producing a compound represented by the following Structural Formula (1), the method including:

introducing a benzyl group into trehalose to thereby produce at least one of a compound represented by the following Structural Formula 4a and a compound represented by the following Structural Formula 4a′;

subjecting at least one of the compound represented by the following Structural Formula 4a and the compound represented by the following Structural Formula 4a′ to prenylation to thereby produce at least one of a compound represented by the following Structural Formula 3a and a compound represented by the following Structural Formula 3a′;

subjecting at least one of the compound represented by the following Structural Formula 3a and the compound represented by the following Structural Formula 3a′ to sharpless asymmetric dihydroxylation to thereby produce at least one of a compound represented by the following Structural Formula 2a and a compound represented by the following Structural Formula 2a′; and

allowing at least one of the compound represented by the following Structural Formula 2a and the compound represented by the following Structural Formula 2a′ to react with hydrogen in the presence of a palladium catalyst to thereby produce the compound represented by the following Structural Formula (1):

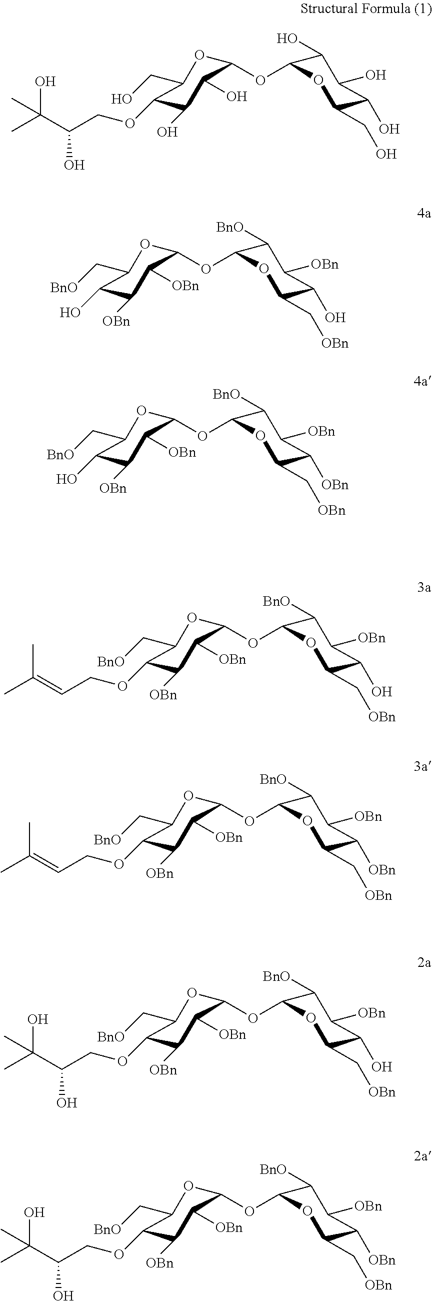

wherein Bn in the Structural Formula 4a, the Structural Formula 4a′, the Structural Formula 3a, the Structural Formula 3a′, the Structural Formula 2a, and the Structural Formula 2a′ denotes a benzyl group.

<2> A method for producing a compound represented by the following Structural Formula (1), the method including:

introducing a benzyl group into trehalose to thereby produce a compound represented by the following Structural Formula 4a′;

allowing the compound represented by the following Structural Formula 4a′ to react with epoxide represented by the following Structural Formula I to thereby produce a compound represented by the following Structural Formula 2a″; and

allowing the compound represented by the following Structural Formula 2a″ to react with hydrogen in the presence of a palladium catalyst to thereby produce the compound represented by the following Structural Formula (1);

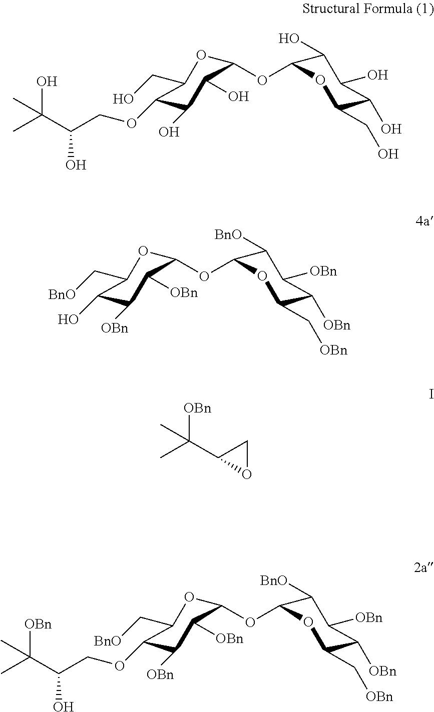

wherein Bn in the Structural Formula 4a′, the Structural Formula I, and the Structural Formula 2a″ denotes a benzyl group.

<3> A method for producing a compound represented by the following Structural Formula (1), the method including:

allowing at least one of a compound represented by the following Structural Formula 2a, a compound represented by the following Structural Formula 2a′, and a compound represented by the following Structural Formula 2a″ to react with hydrogen in the presence of a palladium catalyst:

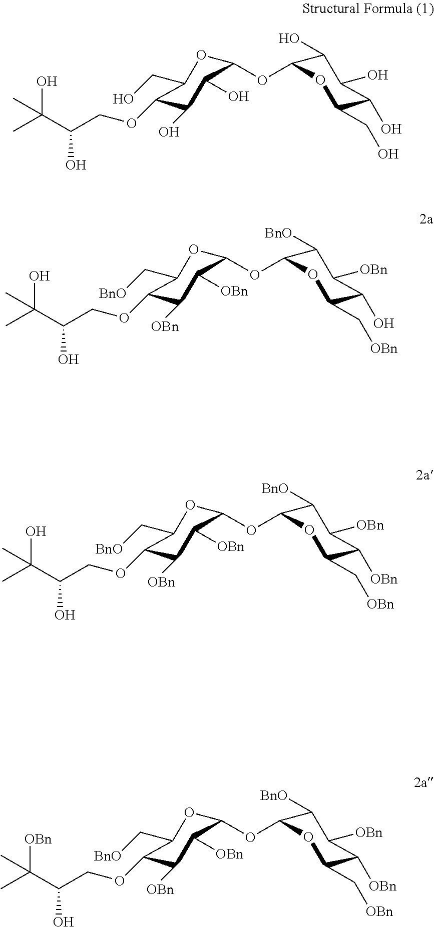

wherein Bn in the Structural Formula 2a, the Structural Formula 2a′, and the Structural Formula 2a″ denotes a benzyl group.

<4> A method for producing at least one of a compound represented by the following Structural Formula 2a and a compound represented by the following Structural Formula 2a′, the method including:

subjecting at least one of a compound represented by the following Structural Formula 3a and a compound represented by the following Structural Formula 3a′ to sharpless asymmetric dihydroxylation:

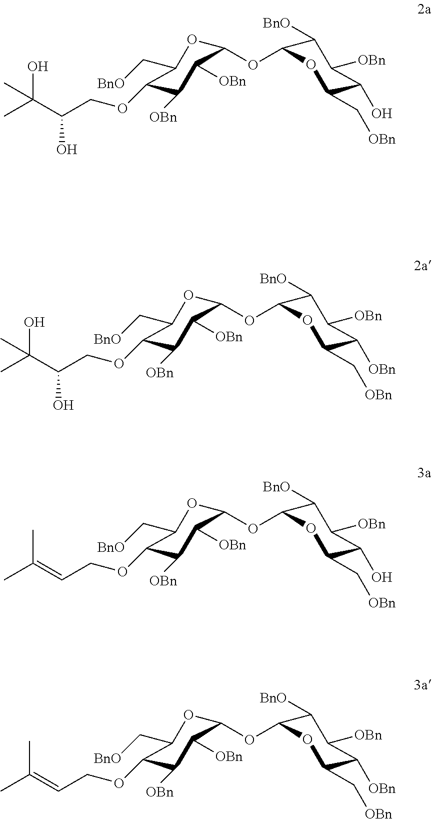

wherein Bn in the Structural Formula 2a, the Structural Formula 2a′, the Structural Formula 3a, and the Structural Formula 3a′ denotes a benzyl group.

<5> A method for producing a compound represented by the following Structural Formula 2a″, the method including:

allowing a compound represented by the following Structural Formula 4a′ to react with epoxide represented by the following Structural Formula I:

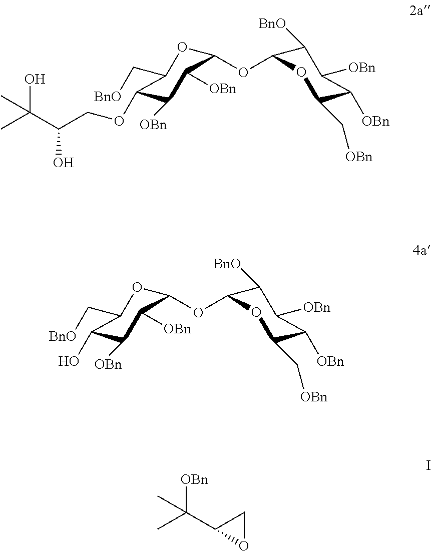

wherein Bn in the Structural Formula 2a″, the Structural Formula 4a′, and the Structural Formula I denotes a benzyl group.

<6> A method for producing a compound represented by the following Structural Formula 3a or the following Structural Formula 3a′, the method including:

subjecting at least one of a compound represented by the following Structural Formula 4a and a compound represented by the following Structural Formula 4a′ to prenylation:

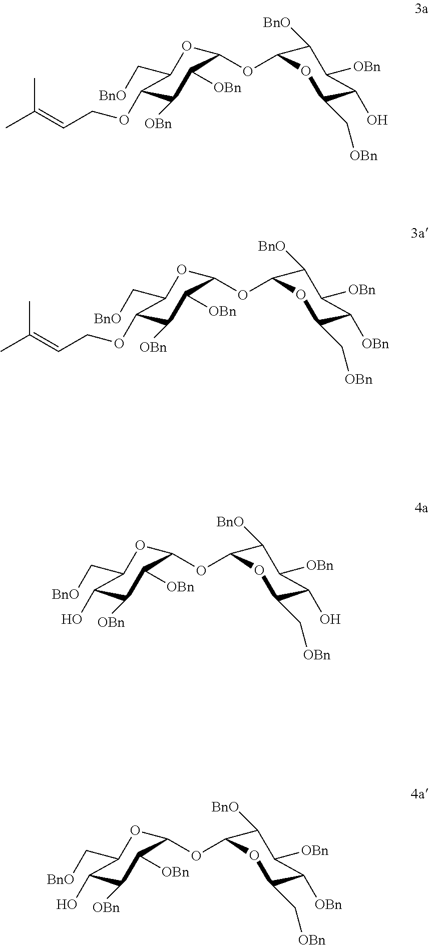

wherein Bn in the Structural Formula 3a, the Structural Formula 3a′, the Structural Formula 4a, and the Structural Formula 4a′ denotes a benzyl group.

<7> A method for producing a compound represented by the following Structural Formula 4a or the following Structural Formula 4a′, the method including:

introducing a benzyl group into trehalose:

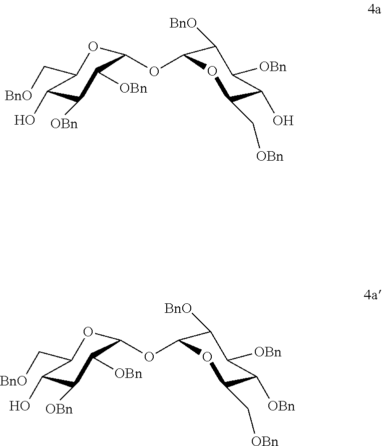

wherein Bn in the Structural Formula 4a and the Structural Formula 4a′ denotes a benzyl group.

<8> A compound represented by the following Structural Formula 2a, the following Structural Formula 2a′, or the following Structural Formula 2a″:

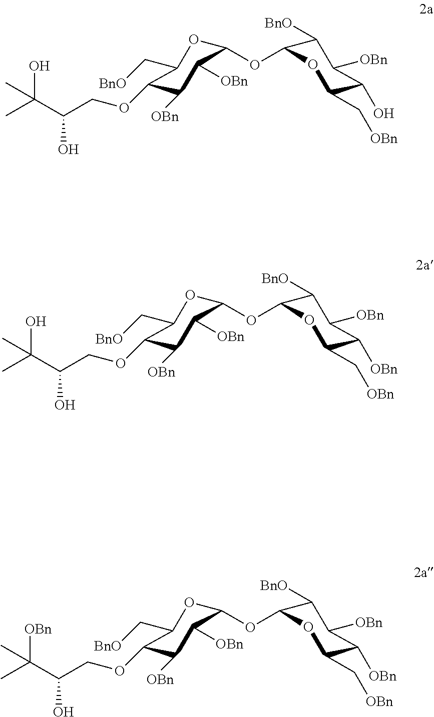

wherein Bn in the Structural Formula 2a, the Structural Formula 2a′, and the Structural Formula 2a″ denotes a benzyl group.

<9> A compound represented by the following Structural Formula 3a or the following Structural Formula 3a′:

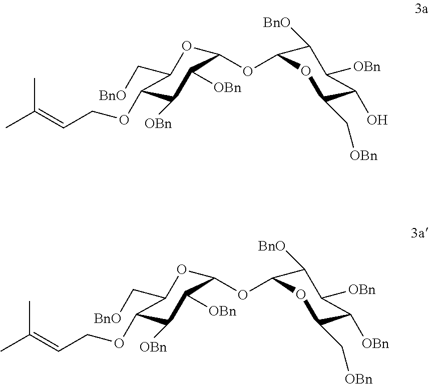

wherein Bn in the Structural Formula 3a and the Structural Formula 3a′ denotes a benzyl group.

<10> A compound represented by the following Structural Formula 4a or the following Structural Formula 4a′

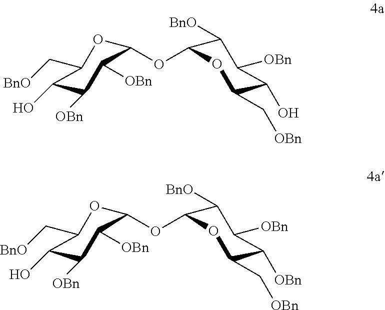

wherein Bn in the Structural Formula 4a and the Structural Formula 4a′ denotes a benzyl group.

The present invention can solve the above existing problems and achieve the above object, and can provide a method for producing lentztrehalose A with high efficiency, an intermediate useful for producing the lentztrehalose A, and a method for producing the intermediate.

### DETAILED DESCRIPTION OF THE INVENTION

(Compound Represented by Structural Formula 4a or Structural Formula 4a′ and Production Method Thereof)

A compound represented by the following Structural Formula 4a or the following Structural Formula 4a′ according to the present invention is an intermediate useful for producing lentztrehalose A, and can be suitably produced by a method for producing a compound represented by the following Structural Formula 4a or the following Structural Formula 4a′ according to the present invention.

The compound represented by the following Structural Formula 4a or the following Structural Formula 4a′ according to the present invention will now be described in conjunction with the method for producing a compound represented by the following Structural Formula 4a or the following Structural Formula 4a′ according to the present invention:

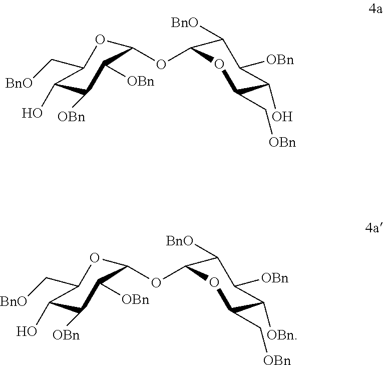

In the Structural Formula 4a and the Structural Formula 4a′, Bn denotes a benzyl group.

<Method for Producing Compound Represented by Structural Formula 4a or Structural Formula 4a′>

The method for producing a compound represented by Structural Formula 4a or Structural Formula 4a′ includes a benzyl group introduction step; and, if necessary, further includes other steps.

### —Benzyl Group Introduction Step—

The benzyl group introduction step is a step of introducing a benzyl group into trehalose to thereby produce the compound represented by Structural Formula 4a or Structural Formula 4a′.

The benzyl group introduction step is not particularly limited and may be appropriately selected depending on the intended purpose, but preferably includes protecting benzylidene and the benzyl group (hereinafter may be referred to as “Bn”) and then regioselectively cleaving acetal.

A compound used for protecting the benzylidene, and an amount and a reaction condition thereof are not particularly limited and may be appropriately selected depending on the intended purpose.

For example, in the case of producing the compound represented by Structural Formula 4a as a main product, the following procedure may be used. The trehalose (6.80 mmol), benzaldehyde dimethylacetal (1 equivalent), and para-toluenesulfonic acid (5 mol %) are dissolved in dimethylformamide, followed by heating to 100° C. and then stirring under reduced pressure of 160 mmHg for 8 hours. Further benzaldehyde dimethylacetal (1 equivalent) is added thereto, and then stirred under reduced pressure of 160 mmHg for 1 hour. Further benzaldehyde dimethylacetal (0.25 equivalents) is added thereto, and then stirred under reduced pressure of 160 mmHg for 4 hours.

For example, in the case of producing the compound represented by Structural Formula 4a′ as a main product, the following procedure may be used. The trehalose (10.4 mmol), benzaldehyde dimethylacetal (1.25 equivalents), and para-toluenesulfonic acid (5.0 mol %) are dissolved in dimethylformamide, and then stirred under reduced pressure of 240 mmHg for 20 hours.

A compound used for protecting the Bn, and an amount and a reaction condition thereof are not particularly limited and may be appropriately selected depending on the intended purpose.

For example, in the case of producing the compound represented by Structural Formula 4a as a main product, the following procedure may be used. A benzylidene-protected product is dissolved in tetrahydrofuran, followed by adding sodium hydride (10 equivalents), tetrabutylammonium iodide (7 mol %), and benzyl bromide (6 equivalents) thereto sequentially under ice cooling and then stirring at room temperature for 45 hours.

For example, in the case of producing the compound represented by Structural Formula 4a′ as a main product, the following procedure may be used. Sodium hydride (15 equivalents), tetrabutylammonium iodide (7 mol %), and benzyl bromide (9 equivalents) are added to a benzylidene-protected mixed solution sequentially under ice cooling, and then stirred at room temperature for 12 hours.

A compound used for regioselectively cleaving acetal, and an amount and a reaction condition thereof are not particularly limited and may be appropriately selected depending on the intended purpose.

For example, in the case of producing the compound represented by Structural Formula 4a as a main product, the following procedure may be used. A Bn-protected product is dissolved in methylene chloride, followed by adding at 0° C. triethylsilane (15 equivalents) and trifluoroacetic acid (15 equivalents) thereto and then stirring at the same temperature for 3 hours.

For example, in the case of producing the compound represented by Structural Formula 4a′ as a main product, the following procedure may be used. A Bn-protected product is dissolved in methylene chloride, followed by adding at 0° C. triethylsilane (7.5 equivalents) and trifluoroacetic acid (7.5 equivalents) thereto and then stirring at the same temperature for 2.5 hours.

### —Other Steps—

The other steps are not particularly limited and may be appropriately selected depending on the intended purpose so long as they do not impair the effect of the present invention. Examples thereof include a trehalose preparation step and a purification step.

A method for preparing the trehalose is not particularly limited and may be appropriately selected from those known in the art. The trehalose may be a commercially available product.

A method for purifying the compound represented by Structural Formula 4a or Structural Formula 4a′ is not particularly limited and may be appropriately selected from those known in the art.

<Compound Represented by Structural Formula 4a or Structural Formula 4a′>
—Compound Represented by Structural Formula 4a—

Results of a melting point, a specific rotation, a ¹H NMR spectrum, a ¹³C NMR spectrum, and HRMS (ESI) analysis of the compound represented by Structural Formula 4a are as follows.

Melting point: 101° C. to 103° C. (dec.).

Specific rotation: [α]²⁵_{D}=79 (c=1.3, CHCl₃).

¹H NMR (400 MHz, CDCl₃): δ 7.38-7.33 (m, 8H), 7.30-7.23 (m, 22H), 5.23 (d, J=3.6 Hz, 2H), 5.00 (d, J=11.4 Hz, 2H), 4.79 (d, J=11.4 Hz, 2H), 4.69 (d, J=12.1 Hz, 2H), 4.63 (d, J=12.1 Hz, 2H), 4.50 (d, J=12.1 Hz, 2H), 4.44 (d, J=12.1 Hz, 2H), 4.13-4.10 (m, 2H), 3.87 (t, J=9.6 Hz, 2H), 3.59 (t, J=9.6 Hz, 2H), 3.56 (dd, J=9.6 Hz, J=3.6 Hz, 2H), 3.53-3.45 (m, 4H), 2.38 (bs, 2H).

¹³C NMR (100 MHz, CDCl₃): δ 138.9, 138.1, 138.0, 128.7, 128.5, 128.5, 128.1, 127.9, 127.8, 127.8, 127.6, 94.3, 81.1, 79.0, 75.4, 73.7, 72.5, 70.8, 70.7, 69.3.

HRMS (ESI) analysis: calcd. for C₅₄H₅₈O₁₁K m/z 921.3611 [M+K]⁺, found 921.3598.

—Compound Represented by Structural Formula 4a′—

Results of a specific rotation, a ¹H NMR spectrum, a ¹³C NMR spectrum, and a HRMS (ESI) analysis of the compound represented by Structural Formula 4a′ are as follows.

Specific rotation: [α]²⁵_{D}=88 (c=1.0, CHCl₃).

¹H NMR (400 MHz, CDCl₃): δ 7.37-7.20 (m, 33H), 7.13-7.11 (m, 2H), 5.24 (d, J=3.4 Hz, 1H), 5.23 (d, J=3.4 Hz, 1H), 5.00 (d, J=11.4 Hz, 1H), 4.99 (d, J=11.0 Hz, 1H), 4.86 (d, J=11.0 Hz, 1H), 4.81 (d, J=10.5 Hz, 1H), 4.79 (d, J=11.4 Hz, 1H), 4.71-4.64 (m, 4H), 4.54 (d, J=12.4 Hz, 1H), 4.50 (d, J=12.4 Hz, 1H), 4.45 (d, J=10.5 Hz, 1H), 4.43 (d, J=12.4 Hz, 1H), 4.37 (d, J=12.4 Hz, 1H), 4.17-4.11 (m, 2H), 4.03 (t, J=9.4 Hz, 1H), 3.87 (t, J=9.4 Hz, 1H), 3.68 (t, J=9.6 Hz, 2H), 3.59 (dd, J=9.6 Hz, J=3.2 Hz, 1H), 3.56 (dd, J=9.8 Hz, J=3.4 Hz, 1H), 3.53-3.44 (m, 3H), 3.36 (d, J=10.1 Hz, 1H), 2.38 (d, J=2.3 Hz, 1H).

¹³C NMR (100 MHz, CDCl₃): δ 138.9, 138.9, 138.4, 138.2, 138.1, 138.0, 137.9, 128.6, 128.5, 128.4, 128.1, 128.0, 128.0, 127.8, 127.8, 127.8, 127.7, 127,7, 127.6, 127.6, 127.4, 94.5, 94.4, 81.9, 81.1, 79.4, 79.1, 77.7, 75.7, 75.4, 75.2, 73.7, 73.6, 72.8, 72.5, 70.9, 70.7, 70.6, 69.2, 68.2.

HRMS (ESI) analysis: calcd. for C₆₁H₆₄O₁₁K m/z 1101.4080 [M+K]⁺, found 1101.4067.

Whether the resultant compound has a structure represented by Structural Formula 4a or Structural Formula 4a′ can be verified by appropriately selected various analytical methods. Examples of the analytical methods include a mass spectrometry, an ultraviolet spectroscopy, an infrared spectroscopy, a proton nuclear magnetic resonance spectrometry, and a carbon-13 nuclear magnetic resonance spectrometry. Note that, although measurement values obtained by the analytical methods have some errors, those skilled in the art can easily identify that the compound has the structure represented by Structural Formula 4a or Structural Formula 4a′.

(Compound Represented by Structural Formula 3a or Structural Formula 3a′ and Production Method Thereof)

A compound represented by the following Structural Formula 3a or the following Structural Formula 3a′ according to the present invention is an intermediate useful for producing the lentztrehalose A, and can be suitably produced by a method for producing a compound represented by the following Structural Formula 3a or the following Structural Formula 3a′ according to the present invention.

The compound represented by the following Structural Formula 3a or the following Structural Formula 3a′ according to the present invention will now be described in conjunction with the method for producing a compound represented by the following Structural Formula 3a or the following Structural Formula 3a′ according to the present invention:

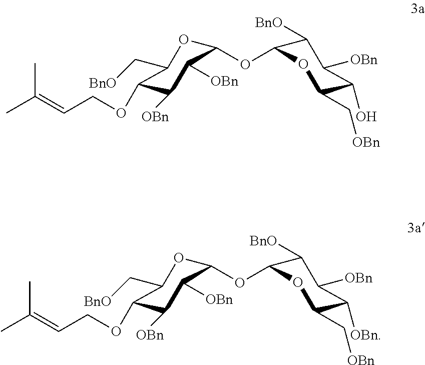

In the Structural Formula 3a and the Structural Formula 3a′, Bn denotes a benzyl group.

<Method for Producing Compound Represented by Structural Formula 3a or Structural Formula 3a′>

The method for producing a compound represented by the Structural Formula 3a or Structural Formula 3a′ includes a prenylation step; and, if necessary, further includes other steps.

### —Prenylation Step—

The prenylation step is a step of subjecting at least one of the compound represented by Structural Formula 4a and the compound represented by Structural Formula 4a′ to prenylation to thereby produce the compound represented by Structural Formula 3a or Structural Formula 3a′.

A compound used for the prenylation, and an amount and a reaction condition thereof are not particularly limited and may be appropriately selected depending on the intended purpose.

For example, in the case of producing the compound represented by Structural Formula 3a, the following procedure may be used. The compound represented by Structural Formula 4a (0.119 mmol) is dissolved in dimethylformamide, followed by adding sodium hydride (2.5 equivalents), tetrabutylammonium iodide (5 mol %), and prenyl bromide (1.1 equivalents) thereto sequentially under ice cooling and then stirring at room temperature for 12 hours.

For example, in the case of producing the compound represented by Structural Formula 3a′, the following procedure may be used. The compound represented by Structural Formula 4a′ (0.109 mmol) is dissolved in dimethylformamide, followed by adding tetrabutylammonium iodide (10 mol %), sodium hydride (2 equivalents), and prenyl bromide (5 equivalents) thereto sequentially under ice cooling and then stirring at room temperature for 12 hours.

### —Other Steps—

The other steps are not particularly limited and may be appropriately selected depending on the intended purpose so long as they do not impair the effect of the present invention. Examples thereof include a production step of at least one of the compound represented by Structural Formula 4a and the compound represented by Structural Formula 4a′ and a purification step.

A method for purifying the compound represented by Structural Formula 3a or Structural Formula 3a′ is not particularly limited and may be appropriately selected from those known in the art.

<Compound Represented by Structural Formula 3a or Structural Formula 3a′>
—Compound Represented by Structural Formula 3a—

Results of a specific rotation, a ¹H NMR spectrum, a ¹³C NMR spectrum, and a HRMS (ESI) analysis of the compound represented by Structural Formula 3a are as follows.

Specific rotation: [α]²⁵_{D}=88 (c=1.0, CHCl₃).

¹H NMR (400 MHz, CDCl₃): δ 7.31-7.12 (m, 30H), 5.19-5.16 (m, 1H), 5.13 (bs, 2H), 4.93 (d, J=11.2 Hz, 1H), 4.90 (d, J=11.2 Hz, 1H), 4.79 (d, J=11.2 Hz, 1H), 4.70 (d, J=11.2 Hz, 1H), 4.62-4.54 (m, 4H), 4.47 (d, J=12.2 Hz, 1H), 4.41 (d, J=12.2 Hz, 1H), 4.36 (d, J=12.2 Hz, 1H), 4.34 (d, J=12.2 Hz, 1H), 4.24-4.19 (m, 1H), 4.08-4.03 (m, 2H), 3.95-3.88 (m, 2H), 3.77 (t, J=9.4 Hz, 1H), 3.59 (t, J=9.4 Hz, 1H), 3.48-3.39 (m, 5H), 3.37-3.12 (m, 2H), 2.30 (bs, 1H), 1.62 (s, 3H), 1.46 (s, 3H).

¹³C NMR (100 MHz, CDCl₃): δ 139.0, 138.9, 138.3, 138.1, 138.0, 138.0, 137.1, 128.6, 128.4, 128.0, 127.9, 127.9, 127.8, 127.7, 127.6, 127.6, 127.6, 127.5, 121.2, 94.7, 94.6, 81.8, 81.3, 79.3, 79.0, 75.6, 75.5, 73.6, 73.6, 72.8, 72.4, 70.9, 70.8, 70.5, 69.7, 69.2, 68.4, 25.9, 18.0.

HRMS (ESI) analysis: calcd. for C₅₉H₆₆O₁₁K m/z 989.4237 [M+K]⁺, found 989.4210.

—Compound Represented by Structural Formula 3a′—

Results of a specific rotation, a ¹H NMR spectrum, a ¹³C NMR spectrum, and a HRMS (ESI) analysis of the compound represented by Structural Formula 3a′ are as follows.

Specific rotation: [α]²⁵_{D}=87 (c=1.6, CHCl₃).

¹H NMR (400 MHz, CDCl₃): δ 7.38-7.20 (m, 33H), 7.12-7.09 (m, 2H), 5.26-5.24 (m, 1H), 5.21-5.20 (m, 2H), 4.99 (d, J=10.8 Hz, 1H), 4.97 (d, J=11.0 Hz, 1H), 4.86 (d, J=10.8 Hz, 1H), 4.84 (d, J=11.0 Hz, 1H), 4.80 (d, J=10.5 Hz, 1H), 4.70-4.62 (m, 4H), 4.56 (d, J=12.1 Hz, 1H), 4.54 (d, J=12.2 Hz, 1H), 4.44 (d, J=12.1 Hz, 1H), 4.43 (d, J=10.5 Hz, 1H), 4.35 (d, J=12.2 Hz, 1H), 4.33-4.27 (m, 1H), 4.17-4.13 (m, 2H), 3.99 (t, J=9.4 Hz, 2H), 3.96 (t, J=9.4 Hz, 1H), 3.69-3.65 (m, 1H), 3.58-3.47 (m, 5H), 3.41 (d, J=10.8 Hz, 1H), 3.33 (d, J=10.8 Hz, 1H), 1.69 (s, 3H), 1.53 (s, 3H).

¹³C NMR (100 MHz, CDCl₃): δ 139.1, 139.0, 138.4, 138.4, 138.3, 138.0, 138.0, 137.2, 128.5, 128.5, 128.4, 128.4, 128.1, 128.1, 128.0, 128.0, 127.8, 127.7, 127.6, 127.6, 127.6, 127.5, 121.3, 94.8, 82.0, 81.8, 79.4, 79.4, 77.7, 77.5, 77.4, 75.8, 75.7, 75.2, 73.6, 72.8, 72.7, 70.8, 70.6, 69.7, 68.4, 68.1, 25.9, 18.1.

HRMS (ESI) analysis: calcd. for C₆₆H₇₂O₁₁Na m/z 1063.4967 [M+Na]⁺, found 1063.4945.

Whether the resultant compound has a structure represented by Structural Formula 3a or Structural Formula 3a′ can be verified by appropriately selected various analytical methods. Examples of the analytical methods include a mass spectrometry, an ultraviolet spectroscopy, an infrared spectroscopy, a proton nuclear magnetic resonance spectrometry, and a carbon-13 nuclear magnetic resonance spectrometry. Note that, although measurement values obtained by the analytical methods have some errors, those skilled in the art can easily identify that the compound has the structure represented by Structural Formula 3a or Structural Formula 3a′.

(Compound Represented by Structural Formula 2a, Structural Formula 2a′, or Structural Formula 2a″ and Production Method Thereof)

A compound represented by the following Structural Formula 2a, the following Structural Formula 2a′, or the following Structural Formula 2a″ according to the present invention is an intermediate useful for producing the lentztrehalose A, and can be suitably produced by a method for producing at least one of a compound represented by the following Structural Formula 2a or a compound represented by the following Structural Formula 2a′, or a method for producing a compound represented by the following Structural Formula 2a″ according to the present invention.

The compound represented by the following Structural Formula 2a, the following Structural Formula 2a′, or the following Structural Formula 2a″ according to the present invention will now be described in conjunction with the method for producing at least one of a compound represented by the following Structural Formula 2a or a compound represented by the following Structural Formula 2a′, or the method for producing a compound represented by the following Structural Formula 2a″ according to the present invention:

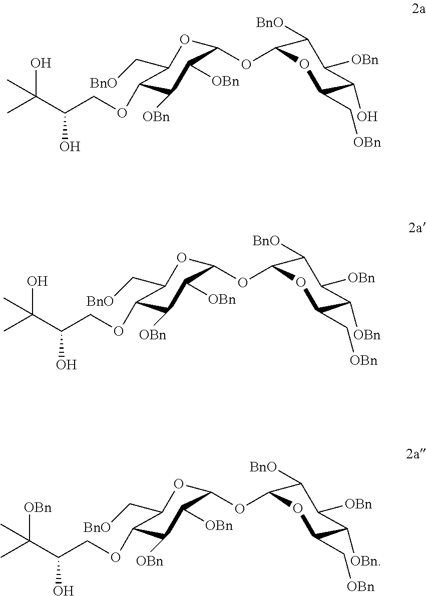

In the Structural Formula 2a, the Structural Formula 2a′, and the Structural Formula 2a″, Bn denotes a benzyl group.

<Method for Producing Compound Represented by Structural Formula 2a or Structural Formula 2a′>

The method for producing a compound represented by Structural Formula 2a or Structural Formula 2a′ includes a sharpless asymmetric dihydroxylation step; and, if necessary, further includes other steps.

### —Sharpless Asymmetric Dihydroxylation Step—

The sharpless asymmetric dihydroxylation step is a step of subjecting at least one of the compound represented by Structural Formula 3a and the compound represented by Structural Formula 3a′ to sharpless asymmetric dihydroxylation to thereby produce the compound represented by Structural Formula 2a or Structural Formula 2a′.

A compound used for the sharpless asymmetric dihydroxylation, and an amount and a reaction condition thereof are not particularly limited and may be appropriately selected depending on the intended purpose.

For example, in the case of producing the compound represented by Structural Formula 2a, the following procedure may be used. The compound represented by Structural Formula 3a (45 μmol) is dissolved in a mixed solvent of t-butanol, water, and acetone (1:1:1), and then stirred with AD-mix-α (118 mg), bis(dihydroquininyl)phthalazine (0.25 equivalents), and methane sulfonamide (2 equivalents) at 0° C. for 66 hours.

For example, in the case of producing the compound represented by Structural Formula 2a′, the following procedure may be used. The compound represented by Structural Formula 3a′ (45 μmol) is dissolved in a mixed solvent of t-butanol, water, and acetone (1:1:1), and then stirred with AD-mix-α (124 mg), bis(dihydroquininyl)phthalazine (0.25 equivalents), and methane sulfonamide (2 equivalents) at 0° C. for 72 hours.

### —Other Steps—

The other steps are not particularly limited and may be appropriately selected depending on the intended purpose so long as they do not impair the effect of the present invention. Examples thereof include a production step of at least one of the compound represented by Structural Formula 3a and the compound represented by Structural Formula 3a′ and a purification step.

A method for purifying the compound represented by Structural Formula 2a or Structural Formula 2a′ is not particularly limited and may be appropriately selected from those known in the art.

<Compound Represented by Structural Formula 2a or Structural Formula 2a′>
—Compound Represented by Structural Formula 2a—

Results of a specific rotation, a ¹H NMR spectrum, a ¹³C NMR spectrum, and a HRMS (ESI) analysis of the compound represented by Structural Formula 2a are as follows.

Specific rotation: [α]²⁵_{D}=88 (c=1.6, CHCl₃).

¹H NMR (400 MHz, CDCl₃): δ 7.40-7.22 (m, 30H), 5.22 (d, J=3.7 Hz, 1H), 5.20 (d, J=3.9 Hz, 1H), 5.03 (d, J=11.2 Hz, 1H), 5.02 (d, J=11.0 Hz, 1H), 4.83 (d, J=11.2 Hz, 1H), 4.78 (d, J=11.0 Hz, 1H), 4.70 (d, J=11.9 Hz, 1H), 4.70-4.66 (m, 2H), 4.62 (d, J=11.9 Hz, 1H), 4.52 (d, J=12.1 Hz, 1H), 4.51 (d, J=12.1 Hz, 1H), 4.44 (d, J=12.1 Hz, 1H), 4.40 (d, J=12.1 Hz, 1H), 4.12 (br dt, J=9.8 Hz, 3.4 Hz, 1H), 4.04 (br dt, J=10.0 Hz, 2.5 Hz, 1H), 3.95-3.92 (m, 1H), 3.90-3.87 (m, 1H), 3.83 (dd, J=10.0 Hz, 2.3 Hz, 1H), 3.73-3.67 (m, 1H), 3.60-3.56 (m, 2H), 3.55-3.53 (m, 1H), 3.51-3.47 (m, 3H), 3.44-3.39 (m, 2H), 3.33 (dd, J=11.0 Hz, 2.1 Hz, 1H), 3.00 (d, J=3.9 Hz, 1H), 2.44 (d, J=2.5 Hz, 1H), 2.42 (bs, 1H), 1.10 (s, 3H), 0.99 (s, 3H).

¹³C NMR (100 MHz, CDCl₃): δ 138.8, 138.5, 138.1, 138.0, 137.6, 128.7, 128.5, 128.5, 128.5, 128.1, 128.0, 127.9, 127.9, 127.8, 127.8, 127.8, 127.6, 127.4, 94.4, 94.2, 81.3, 81.2, 79.6, 79.2, 78.1, 76.3, 75.7, 75.4, 73.8, 73.7, 73.7, 72.7, 72.7, 71.3, 70.9, 70.7, 70.6, 69.3, 68.5, 26.6, 24.6.

HRMS (ESI) analysis: calcd. for C₅₉H₆₈O₁₃Na m/z 1007.4552 [M+Na]⁺, found 1007.4525.

—Compound Represented by Structural Formula 2a′—

Results of a ¹H NMR spectrum, a ¹³C NMR spectrum, and a HRMS (ESI) analysis of the compound represented by Structural Formula 2a′ are as follows.

¹H NMR (400 MHz, CDCl₃): δ 7.38-7.13 (m, 33H), 7.12-7.09 (m, 2H), 5.20 (d, J=3.4 Hz, 1H), 5.19 (d, J=3.4 Hz, 1H), 5.02 (d, J=10.8 Hz, 1H), 5.01 (d, J=10.8 Hz, 1H), 4.89 (d, J=10.8 Hz, 1H), 4.81 (d, J=10.5 Hz, 1H), 4.77 (d, J=10.8 Hz, 1H), 4.72 (d, J=12.1 Hz, 1H), 4.68-4.61 (m, 2H), 4.64 (d, J=12.1 Hz, 1H), 4.54 (d, J=12.1 Hz, 1H), 4.53 (d, J=12.1 Hz, 1H), 4.45 (d, J=10.5 Hz, 1H), 4.40 (d, J=12.1 Hz, 1H), 4.37 (d, J=12.1 Hz, 1H), 4.15 (br dt, J=9.8 Hz, 3.4 Hz, 1H), 4.14-06 (m, 2H), 3.91 (t, J=9.4 Hz, 1H), 3.82 (dd, J=9.4 Hz, 2.3 Hz, 1H), 3.69 (t, J=9.4 Hz, 1H), 3.60 (dd, J=9.6 Hz, 3.4 Hz, 1H), 3.54 (dd, J=9.4 Hz, 3.6 Hz, 1H), 3.52 (dd, J=9.4 Hz, 3.6 Hz, 1H), 3.49 (dd, J=9.6 Hz, 8.0 Hz, 2H), 3.44-3.41 (m, 1H), 3.41-3.39 (m, 1H), 3.37 (dd, J=10.8 Hz, 1.8 Hz, 1H), 3.32 (dd, J=10.8 Hz, 2.3 Hz, 1H), 3.00 (d, J=3.9 Hz, 1H), 2.40 (s, 1H), 1.10 (s, 3H), 0.98 (s, 3H).

¹³C NMR (100 MHz, CDCl₃): δ 138.9, 138.6, 138.4, 138.3, 138.1, 137.9, 137.7, 128.5, 128.5, 128.5, 128.1, 128.1, 128.0, 127.9, 127.9, 127.8, 127.8, 127.8, 127.8, 127.7, 127.5, 127.5, 94.6, 94.4, 82.0, 81.3, 79.6, 79.6, 78.2, 77.8, 76.3, 75.7, 75.7, 75.3, 73.8, 73.7, 73.6, 73.0, 72.6, 71.3, 70.8, 70.5, 68.5, 68.2, 26.6, 24.6.

HRMS (ESI) analysis: calcd. for C₆₆H₇₄O₁₃Na m/z 1097.5022 [M+Na]⁺, found 1097.4999.

Whether the resultant compound has a structure represented by Structural Formula 2a or Structural Formula 2a′ can be verified by appropriately selected various analytical methods. Examples of the analytical methods include a mass spectrometry, an ultraviolet spectroscopy, an infrared spectroscopy, a proton nuclear magnetic resonance spectrometry, and a carbon-13 nuclear magnetic resonance spectrometry. Note that, although measurement values obtained by the analytical methods have some errors, those skilled in the art can easily identify that the compound has the structure represented by Structural Formula 2a or Structural Formula 2a′.

<Method for Producing Compound Represented by Structural Formula 2a″>

A method for producing the compound represented by Structural Formula 2a″ includes an epoxide ring-opening step; and, if necessary, further includes other steps.

### —Epoxide Ring-Opening Step—

The epoxide ring-opening step is a step of allowing the compound represented by Structural Formula 4a′ to react with epoxide represented by the following Structural Formula I to thereby produce the compound represented by Structural Formula 2a″:

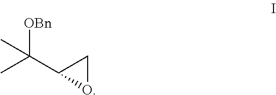

In the Structural Formula I, Bn denotes a benzyl group.

A compound used for the epoxide ring-opening, and an amount and a reaction condition thereof are not particularly limited and may be appropriately selected depending on the intended purpose.

For example, the following procedure may be used. The compound represented by Structural Formula 4a′ (25.1 μmol) is dissolved in dimethylformamide, followed by adding sodium hydride (2.5 equivalents), 15-crown-5 (2.6 equivalents), and the epoxide represented by Structural Formula I (2.5 equivalents) thereto sequentially under ice cooling and then stirring at 70° C. for 18 hours.

### —Other Steps—

The other steps are not particularly limited and may be appropriately selected depending on the intended purpose so long as they do not impair the effect of the present invention. Examples thereof include a production step of the compound represented by Structural Formula 4a′ and a purification step.

A method for purifying the compound represented by Structural Formula 2a″ is not particularly limited and may be appropriately selected from those known in the art.

<Compound Represented by Structural Formula 2a″>

Results of a ¹H NMR spectrum and a HRMS (ESI) analysis of the compound represented by Structural Formula 2a″ are as follows.

¹H NMR (400 MHz, CDCl₃): δ 7.41-7.24 (m, 36H), 7.14-7.11 (m, 2H), 5.21 (d, J=3.4 Hz, 2H), 5.02 (d, J=11.0 Hz, 1H), 5.00 (d, J=10.5 Hz, 1H), 4.88 (d, J=11.0 Hz, 1H), 4.87 (d, J=10.5 Hz, 1H), 4.82 (d, J=10.5 Hz, 1H), 4.71-4.64 (m, 4H), 4.55 (d, J=12.1 Hz, 1H), 4.49 (d, J=11.9 Hz, 1H), 4.47 (d, J=10.5 Hz, 1H), 4.44-4.42 (m, 2H), 4.42 (d, J=11.9 Hz, 1H), 4.37 (d, J=12.1 Hz, 1H), 4.18-4-4.06 (m, 2H), 4.03 (t, J=9.4 Hz, 1H), 4.00 (t, J=9.4 Hz, 1H), 3.97-3.93 (m, 1H), 3.85 (dd, J=10.3 Hz, 1.6 Hz, 1H), 3.75-3.63 (m, 3H), 3.58-3.46 (m, 5H), 3.42-3.34 (m, 2H), 1.15 (s, 3H), 1.11 (s, 3H).

HRMS (ESI) analysis: calcd. for C₇₃H₈₀O₁₃Na m/z 1187.5491 [M+Na]⁺, found 1187.5476.

Whether the resultant compound has a structure represented by Structural Formula 2a″ can be verified by appropriately selected various analytical methods. Examples of the analytical methods include a mass spectrometry, an ultraviolet spectroscopy, an infrared spectroscopy, a proton nuclear magnetic resonance spectrometry, and a carbon-13 nuclear magnetic resonance spectrometry. Note that, although measurement values obtained by the analytical methods have some errors, those skilled in the art can easily identify that the compound has the structure represented by Structural Formula 2a″.

### (Method for Producing Lentztrehalose A)

An aspect of a method for producing lentztrehalose A according to the present invention includes an aspect in which at least one of the compound represented by Structural Formula 2a, the compound represented by Structural Formula 2a′, and the compound represented by Structural Formula 2a″ is used as a starting material (hereinafter may be referred to as “first aspect”) or an aspect in which the trehalose is used as a starting material (hereinafter may be referred to as “second aspect”).

### <First Aspect>

The first aspect includes a debenzylation step; and, if necessary, further includes other steps.

### —Debenzylation Step—

The debenzylation step is a step of allowing at least one of the compound represented by Structural Formula 2a, the compound represented by Structural Formula 2a′, and the compound represented by Structural Formula 2a″ to react with hydrogen in the presence of a palladium catalyst to thereby produce lentztrehalose A.

A compound used for the debenzylation, and an amount and a reaction condition thereof are not particularly limited and may be appropriately selected depending on the intended purpose so long as the compound is allowed to react with hydrogen in the presence of a palladium catalyst.

For example, in the case of using the compound represented by Structural Formula 2a, the following procedure may be used. The compound represented by Structural Formula 2a (28.4 μmol) is dissolved in methanol (2 mL), followed by adding 10% palladium on carbon (Pd/C) (20 mg) thereto and then stirring under a hydrogen atmosphere for 24 hours.

For example, in the case of using the compound represented by Structural Formula 2a′, the following procedure may be used. The compound represented by Structural Formula 2a′ (28.4 μmol) is dissolved in methanol (1.5 mL), followed by adding 10% Pd/C (15 mg) thereto and then stirring under a hydrogen atmosphere for 24 hours.

For example, in the case of using the compound represented by Structural Formula 2a″, the following procedure may be used. The compound represented by Structural Formula 2a″ (7.29 μmol) is dissolved in methanol (1 mL), followed by adding 10% Pd/C (7.0 mg) thereto and then stirring under a hydrogen atmosphere for 24 hours.

### —Other Steps—

The other steps are not particularly limited and may be appropriately selected depending on the intended purpose so long as they do not impair the effect of the present invention. Examples thereof include a production step of at least one of the compound represented by Structural Formula 2a, the compound represented by Structural Formula 2a′, and the compound represented by Structural Formula 2a″ and a purification step.

A method for purifying the lentztrehalose A is not particularly limited and may be appropriately selected from those known in the art.

### <Second Aspect>

The second aspect includes an aspect including a prenylation step and a sharpless asymmetric dihydroxylation step (hereinafter may be referred to as “second aspect-1”) or an aspect including an epoxide ring-opening step (hereinafter may be referred to as “second aspect-2”).

### <<Second Aspect-1>>

The second aspect-1 includes a benzyl group introduction step, a prenylation step, a sharpless asymmetric dihydroxylation step, and a debenzylation step; and, if necessary, further includes other steps.

### —Benzyl Group Introduction Step—

The benzyl group introduction step is a step of introducing a benzyl group into trehalose to thereby produce at least one of the compound represented by Structural Formula 4a and the compound represented by Structural Formula 4a′, and is the same as those described under the heading —Benzyl group introduction step— in <Method for producing compound represented by Structural Formula 4a or Structural Formula 4a′>.

### —Prenylation Step—

The prenylation step is a step of subjecting at least one of the compound represented by Structural Formula 4a and the compound represented by Structural Formula 4a′ to prenylation to thereby produce the compound represented by Structural Formula 3a or the compound represented by Structural Formula 3a′, and is the same as those described under the heading —Prenylation step— in <Method for producing compound represented by Structural Formula 3a or Structural Formula 3a′>.

### —Sharpless Asymmetric Dihydroxylation Step—

The sharpless asymmetric dihydroxylation step is a step of subjecting at least one of the compound represented by Structural Formula 3a and the compound represented by Structural Formula 3a′ to sharpless asymmetric dihydroxylation to thereby produce at least one of the compound represented by Structural Formula 2a and the compound represented by Structural Formula 2a′, and is the same as those described under the heading —Sharpless asymmetric dihydroxylation step— in <Method for producing compound represented by Structural Formula 2a or Structural Formula 2a′>.

### —Debenzylation Step—

The debenzylation step is a step of allowing at least one of the compound represented by Structural Formula 2a and the compound represented by Structural Formula 2a′ to react with hydrogen in the presence of a palladium catalyst to thereby produce the compound represented by Structural Formula (1), and is the same as those described under the heading —Debenzylation step— in <First aspect>.

### —Other Steps—

The other steps are not particularly limited and may be appropriately selected depending on the intended purpose so long as they do not impair the effect of the present invention. Examples thereof include a trehalose preparation step and a lentztrehalose A purification step.

### <<Second Aspect-2>>

The second aspect-2 includes a benzyl group introduction step, an epoxide ring-opening step, and a debenzylation step; and, if necessary, further includes other steps.

### —Benzyl Group Introduction Step—

The benzyl group introduction step is a step of introducing a benzyl group into trehalose to thereby produce the compound represented by Structural Formula 4a′, and is the same as those described under the heading —Benzyl group introduction step— in <Method for producing compound represented by Structural Formula 4a or

Structural Formula 4a′>.

### —Epoxide Ring-Opening Step—

The epoxide ring-opening step is a step of allowing the compound represented by Structural Formula 4a′ to react with the epoxide represented by Structural Formula I to thereby produce the compound represented by Structural Formula 2a″, and is the same as those described under the heading —Epoxide ring-opening step— in <Method for producing compound represented by Structural Formula 2a″>.

### —Debenzylation Step—

The debenzylation step is a step of allowing the compound represented by Structural Formula 2a″ to react with hydrogen in the presence of a palladium catalyst to thereby produce the compound represented by Structural Formula (1), and is the same as those described under the heading —Debenzylation step— in <First aspect>.

### —Other Steps—

The other steps are not particularly limited and may be appropriately selected depending on the intended purpose so long as they do not impair the effect of the present invention. Examples thereof include a trehalose preparation step and a lentztrehalose A purification step.

### <Compound Represented by Structural Formula (1)>

Results of a ¹H NMR spectrum, a ¹³C NMR spectrum, and a HRMS (ESI) analysis of the compound represented by Structural Formula (1) are as follows.

¹H NMR (400 MHz, CD₃OD): δ 5.10 (d, J=3.8 Hz, 1H), 5.08 (d, J=3.9 Hz, 1H), 4.02 (dd, J=10.5 Hz, 2.7 Hz, 1H), 3.90 (t, J=9.4 Hz, 1H), 3.84 (br dt, J=9.4 Hz, 2.8 Hz, 1H), 3.80-3.76 (m, 5H), 3.67 (dd, J=11.7 Hz, 4.4 Hz, 1H), 3.66-3.63 (m, 1H), 3.54 (dd, J=7.8 Hz, 2.5 Hz, 1H), 3.49 (dd, J=9.6 Hz, 3.8 Hz, 1H), 3.46 (dd, J=9.6 Hz, 3.8 Hz, 1H), 3.31 (t, J=9.4 Hz, 1H), 3.28 (t, J=9.4 Hz, 1H), 1.19 (s, 3H), 1.17 (s, 3H).

¹³C NMR (100 MHz, CDCl₃): δ 95.1, 95.0, 80.4, 78.1, 74.7, 74.5, 74.4, 73.8, 73.4, 73.2, 72.8, 72.7, 71.9, 62.6, 62.1, 26.5, 25.4.

HRMS (ESI) analysis: calcd. for C₁₇H₃₂O₁₃Na m/z 467.1735 [M+Na]⁺, found 467.1725.

Whether the resultant compound has a structure represented by Structural Formula (1) can be verified by appropriately selected various analytical methods. Examples of the analytical methods include a mass spectrometry, an ultraviolet spectroscopy, an infrared spectroscopy, a proton nuclear magnetic resonance spectrometry, and a carbon-13 nuclear magnetic resonance spectrometry. Note that, although measurement values obtained by the analytical methods have some errors, those skilled in the art can easily identify that the compound has the structure represented by Structural Formula (1).

### EXAMPLES

The present invention will now be described in detail with reference to Examples of the present invention, but is not limited thereto in any way.

Note that, in the following Examples, “PhCH(OMe)₂” denotes benzaldehyde dimethylacetal, “p-TsOH” denotes para-toluenesulfonic acid, “DMF” denotes dimethylformamide, “BnBr” denotes benzyl bromide, “NaH” denotes sodium hydride, “TBAI” denotes tetrabutylammonium iodide, “THF” denotes tetrahydrofuran, “Et₃SiH” denotes triethylsilane, “TFA” denotes trifluoroacetic acid, “CH₂Cl₂” denotes methylene chloride, “Bn” denotes a benzyl group, “(DHQ)₂PHAL” denotes bis(dihydroquininyl)phthalazine, “MeSO₂NH₂” denotes methane sulfonamide, “t-BuOH” denotes t-butanol, “Pd/C” denotes palladium on carbon, and “MeOH” denotes methanol.

### Example 1

### Synthesis of Lentztrehalose A Via Compound Represented by Structural Formula 4a

<Synthesis of Compound Represented by Structural Formula 4a and Compound Represented by Structural Formula 4a′>

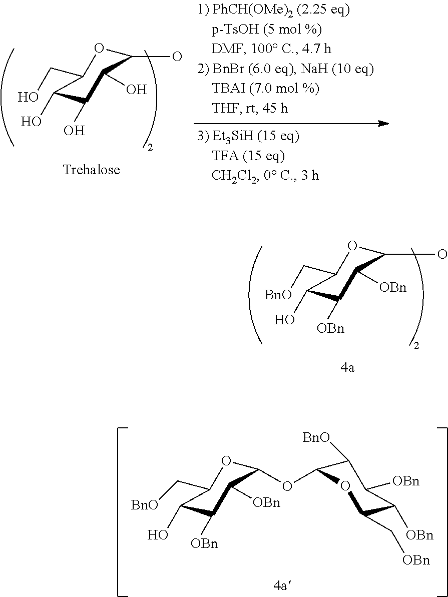

1) Trehalose (2.33 g, 6.80 mmol), benzaldehyde dimethylacetal (1.02 mL, 1 equivalent), and p-TsOH (59 mg, 5 mol %) were dissolved in DMF (30 mL) and then heated to 100° C. Twenty minutes later, the reaction system was reduced in pressure to 160 mmHg and stirred for 20 min. Benzaldehyde dimethylacetal (1.02 mL, 1.0 equivalent) was added thereto, and then stirred under reduced pressure of 160 mmHg and the same condition for 1 hour. Further, benzaldehyde dimethylacetal (0.25 mL, 0.25 equivalents) was added thereto, and continued to stir under reduced pressure of 160 mmHg for 4 hours. The reaction system was cooled to room temperature, concentrated under reduced pressure, and then neutralized with triethylamine. The resultant crude product was used in the next reaction without further purification.

2) The whole quantity of the crude product was dissolved in tetrahydrofuran (70 mL), followed by adding sodium hydride (dispersed in 60% liquid paraffin (the same applied to sodium hydride described below)) (2.72 g, 10 equivalents), TBAI (176 mg, 7 mol %), and benzyl bromide (4.85 mL, 6 equivalents) thereto sequentially under ice cooling and then stirring at room temperature for 45 hours. The resultant reaction liquid was poured into ice-water (40 mL) and quenched, from which an organic layer was separated. An aqueous layer was extracted with ether (30 mL×3). The combined organic layer was washed with aqueous hydrochloric acid solution (1 N, 20 mL) and saturated aqueous sodium bicarbonate solution (40 mL), and then dried over sodium sulfate. The solvent was distilled off, followed by purifying by silica gel column chromatography (hexane:ethyl acetate=6:1) to thereby obtain a benzyl protected product (4.89 g).

3) The benzyl protected product was dissolved in methylene chloride (300 mL), followed by adding at 0° C. triethylsilane (11.7 mL, 15 equivalents) and trifluoroacetic acid (5.61 mL, 15 equivalents) thereto and then stirring at the same temperature for 3 hours. Saturated aqueous NaHCO₃ solution (100 mL) was added thereto, and then stirred for 5 min. Then, an organic layer was separated and washed with saturated aqueous sodium chloride solution. An aqueous layer was extracted with methylene chloride (100 mL). The resultant organic layer was washed with saturated aqueous sodium chloride solution and dried over sodium sulfate. Then, the solvent was distilled off. The resultant crude syrup was purified by silica gel column chromatography (hexane:ethyl acetate=4:1 to 3:1) to thereby obtain a compound represented by Structural Formula 4a (3.11 g, 3.52 mmol, 52% in 3 steps) as white powder and a compound represented by Structural Formula 4a′ (337 mg, 0.346 mmol, 5% in 3 steps) as colorless syrup.

—Compound Represented by Structural Formula 4a—

Results of a melting point, a specific rotation, a ¹H NMR spectrum, a ¹³C NMR spectrum, and a HRMS (ESI) analysis of the compound represented by Structural Formula 4a are as follows.

Melting point: 101° C. to 103° C. (dec.).

Specific rotation: [α]²⁵_{D}=79 (c=1.3, CHCl₃).

¹H NMR (400 MHz, CDCl₃): δ 7.38-7.33 (m, 8H), 7.30-7.23 (m, 22H), 5.23 (d, J=3M Hz, 2H), 5.00 (d, J=11.4 Hz, 2H), 4.79 (d, J=11.4 Hz, 2H), 4.69 (d, J=12.1 Hz, 2H), 4.63 (d, J=12.1 Hz, 2H), 4.50 (d, J=12.1 Hz, 2H), 4.44 (d, J=12.1 Hz, 2H), 4.13-4.10 (m, 2H), 3.87 (t, J=9.6 Hz, 2H), 3.59 (t, J=9.6 Hz, 2H), 3.56 (dd, J=9.6 Hz, J=3.6 Hz, 2H), 3.53-3.45 (m, 4H), 2.38 (bs, 2H).

¹³C NMR (100 MHz, CDCl₃): δ 138.9, 138.1, 138.0, 128.7, 128.5, 128.5, 128.1, 127.9, 127.8, 127.8, 127.6, 94.3, 81.1, 79.0, 75.4, 73.7, 72.5, 70.8, 70.7, 69.3.

HRMS (ESI) analysis: calcd. for C₅₄H₅₈O₁₁K m/z 921.3611 [M+K]⁺, found 921.3598.

—Compound Represented by Structural Formula 4a′—

Results of a specific rotation, a ¹H NMR spectrum, a ¹³C NMR spectrum, and a HRMS (ESI) analysis of the compound represented by Structural Formula 4a′ are as follows.

Specific rotation: [α]²⁵_{D}=88 (c=1.0, CHCl₃).

¹H NMR (400 MHz, CDCl₃): δ 7.37-7.20 (m, 33H), 7.13-7.11 (m, 2H), 5.24 (d, J=3.4 Hz, 1H), 5.23 (d, J=3.4 Hz, 1H), 5.00 (d, J=11.4 Hz, 1H), 4.99 (d, J=11.0 Hz, 1H), 4.86 (d, J=11.0 Hz, 1H), 4.81 (d, J=10.5 Hz, 1H), 4.79 (d, J=11.4 Hz, 1H), 4.71-4.64 (m, 4H), 4.54 (d, J=12.4 Hz, 1H), 4.50 (d, J=12.4 Hz, 1H), 4.45 (d, J=10.5 Hz, 1H), 4.43 (d, J=12.4 Hz, 1H), 4.37 (d, J=12.4 Hz, 1H), 4.17-4.11 (m, 2H), 4.03 (t, J=9.4 Hz, 1H), 3.87 (t, J=9.4 Hz, 1H), 3.68 (t, J=9.6 Hz, 2H), 3.59 (dd, J=9.6 Hz, J=3.2 Hz, 1H), 3.56 (dd, J=9.8 Hz, J=3.4 Hz, 1H), 3.53-3.44 (m, 3H), 3.36 (d, J=10.1 Hz, 1H), 2.38 (d, J=2.3 Hz, 1H).

¹³C NMR (100 MHz, CDCl₃): δ 138.9, 138.9, 138.4, 138.2, 138.1, 138.0, 137.9, 128.6, 128.5, 128.4, 128.1, 128.0, 128.0, 127.8, 127.8, 127.8, 127.7, 127,7, 127.6, 127.6, 127.4, 94.5, 94.4, 81.9, 81.1, 79.4, 79.1, 77.7, 75.7, 75.4, 75.2, 73.7, 73.6, 72.8, 72.5, 70.9, 70.7, 70.6, 69.2, 68.2.

HRMS (ESI) analysis: calcd. for C₆₄H₆₄O₁₁K m/z 1101.4080 [M+K]⁺, found 1101.4067.

<Synthesis of Compound Represented by Structural Formula 3a>

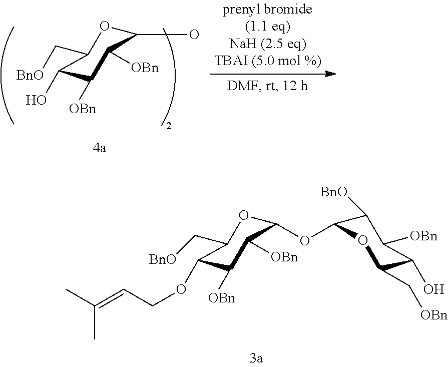

The compound represented by Structural Formula 4a (105 mg, 0.119 mmol) was dissolved in DMF (2 mL), followed by adding sodium hydride (12.0 mg, 2.5 equivalents), TBAI (2.0 mg, 5 mol %), and prenyl bromide (15.1 μL, 1.1 equivalents) thereto sequentially under ice cooling and then stirring at room temperature for 12 hours. Methanol (0.1 mL) was added thereto, and stirred for 5 min. Then, distilled water (10 mL) and methylene chloride (10 mL) were added thereto. An organic layer was separated, washed with aqueous hydrochloric acid solution (1 N, 5 mL) and saturated aqueous sodium bicarbonate solution (5 mL), and dried over sodium sulfate. The solvent was distilled off, followed by purifying by silica gel column chromatography (hexane:ethyl acetate=5:1) to thereby obtain a compound represented by Structural Formula 3a (50.0 mg, 52.6 μmol, yield: 44%). Note that, 27.0 mg (30.6 μmol) of the material was recovered. Taking this into account, the yield was corrected to 65%.

—Compound Represented by Structural Formula 3a—

Results of a specific rotation, a ¹H NMR spectrum, a ¹³C NMR spectrum, and a HRMS (ESI) analysis of the compound represented by Structural Formula 3a are as follows.

Specific rotation: [α]²⁵_{D}=88 (c=1.0, CHCl₃).

¹H NMR (400 MHz, CDCl₃): δ 7.31-7.12 (m, 30H), 5.19-5.16 (m, 1H), 5.13 (bs, 2H), 4.93 (d, J=11.2 Hz, 1H), 4.90 (d, J=11.2 Hz, 1H), 4.79 (d, J=11.2 Hz, 1H), 4.70 (d, J=11.2 Hz, 1H), 4.62-4.54 (m, 4H), 4.47 (d, J=12.2 Hz, 1H), 4.41 (d, J=12.2 Hz, 1H), 4.36 (d, J=12.2 Hz, 1H), 4.34 (d, J=12.2 Hz, 1H), 4.24-4.19 (m, 1H), 4.08-4.03 (m, 2H), 3.95-3.88 (m, 2H), 3.77 (t, J=9.4 Hz, 1H), 3.59 (t, J=9.4 Hz, 1H), 3.48-3.39 (m, 5H), 3.37-3.12 (m, 2H), 2.30 (bs, 1H), 1.62 (s, 3H), 1.46 (s, 3H).

¹³C NMR (100 MHz, CDCl₃): δ 139.0, 138.9, 138.3, 138.1, 138.0, 138.0, 137.1, 128.6, 128.4, 128.0, 127.9, 127.9, 127.8, 127.7, 127.6, 127.6, 127.6, 127.5, 121.2, 94.7, 94.6, 81.8, 81.3, 79.3, 79.0, 75.6, 75.5, 73.6, 73.6, 72.8, 72.4, 70.9, 70.8, 70.5, 69.7, 69.2, 68.4, 25.9, 18.0.

HRMS (ESI) analysis: calcd. for C₅₉H₆₆O₁₁K m/z 989.4237 [M+K]⁺, found 989.4210.

<Synthesis of Compound Represented by Structural Formula 2a>

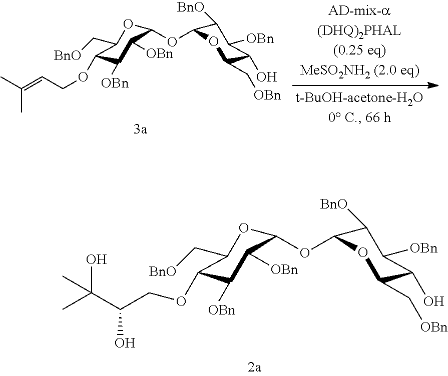

The compound represented by Structural Formula 3a (42.8 mg, 45 μmol) was dissolved in a mixed solvent of t-butanol, water, and acetone (1:1:1), followed by stirring with AD-mix-α (118 mg), (DHQ)₂PHAL (8.8 mg, 0.25 equivalents), and methane sulfonamide (8.5 mg, 2 equivalents) at 0° C. for 66 hours. The reaction system was neutralized by adding sodium sulfite thereto and stirring at 0° C. for 30 min, and then extracted with ethyl acetate. An organic layer was washed with saturated aqueous sodium chloride solution, dried over sodium sulfate, and then concentrated to dryness. A diastereomeric ratio was found to be 9:1 by ¹H NMR. The residue was purified by silica gel column chromatography (hexane:ethyl acetate=3:1) to thereby obtain a compound represented by Structural Formula 2a (44.5 mg, 45 μmol) quantitatively.

—Compound Represented by Structural Formula 2a—

Results of a specific rotation, a ¹H NMR spectrum, a ¹³C NMR spectrum, and a HRMS (ESI) analysis of the compound represented by Structural Formula 2a are as follows.

Specific rotation: [α]²⁵_{D}=88 (c=1.6, CHCl₃).

¹H NMR (400 MHz, CDCl₃): δ 7.40-7.22 (m, 30H), 5.22 (d, J=3.7 Hz, 1H), 5.20 (d, J=3.9 Hz, 1H), 5.03 (d, J=11.2 Hz, 1H), 5.02 (d, J=11.0 Hz, 1H), 4.83 (d, J=11.2 Hz, 1H), 4.78 (d, J=11.0 Hz, 1H), 4.70 (d, J=11.9 Hz, 1H), 4.70-4.66 (m, 2H), 4.62 (d, J=11.9 Hz, 1H), 4.52 (d, J=12.1 Hz, 1H), 4.51 (d, J=12.1 Hz, 1H), 4.44 (d, J=12.1 Hz, 1H), 4.40 (d, J=12.1 Hz, 1H), 4.12 (br dt, J=9.8 Hz, 3.4 Hz, 1H), 4.04 (br dt, J=10.0 Hz, 2.5 Hz, 1H), 3.95-3.92 (m, 1H), 3.90-3.87 (m, 1H), 3.83 (dd, J=10.0 Hz, 2.3 Hz, 1H), 3.73-3.67 (m, 1H), 3.60-3.56 (m, 2H), 3.55-3.53 (m, 1H), 3.51-3.47 (m, 3H), 3.44-3.39 (m, 2H), 3.33 (dd, J=11.0 Hz, 2.1 Hz, 1H), 3.00 (d, J=3.9 Hz, 1H), 2.44 (d, J=2.5 Hz, 1H), 2.42 (bs, 1H), 1.10 (s, 3H), 0.99 (s, 3H).

¹³C NMR (100 MHz, CDCl₃): δ 138.8, 138.5, 138.1, 138.0, 137.6, 128.7, 128.5, 128.5, 128.5, 128.1, 128.0, 127.9, 127.9, 127.8, 127.8, 127.8, 127.6, 127.4, 94.4, 94.2, 81.3, 81.2, 79.6, 79.2, 78.1, 76.3, 75.7, 75.4, 73.8, 73.7, 73.7, 72.7, 72.7, 71.3, 70.9, 70.7, 70.6, 69.3, 68.5, 26.6, 24.6.

HRMS (ESI) analysis: calcd. for C₅₉H₆₈O₁₃Na m/z 1007.4552 [M+Na]⁺, found 1007.4525.

### <Synthesis of Compound Represented by Structural Formula (1)>

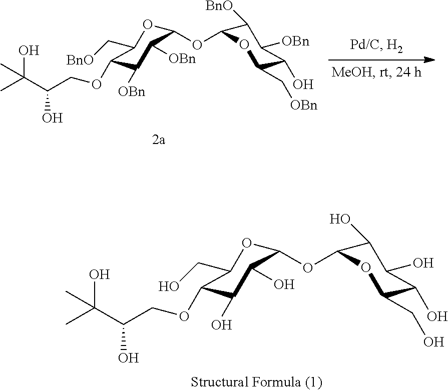

The compound represented by Structural Formula 2a (28.0 mg, 28.4 μmol) was dissolved in methanol (2 mL). To this, was added 10% palladium on carbon (Pd/C) (20 mg), followed by stirring under a hydrogen atmosphere for 24 hours. After filtering the catalyst off through Celite, the resultant filtrate was concentrated to dryness to thereby obtain a compound represented by Structural Formula (1) (lentztrehalose A) (12.6 mg, 28.4 μmol) quantitatively (6 steps, yield: 23%).

### —Compound Represented by Structural Formula (1)—

Results of a ¹H NMR spectrum, a ¹³C NMR spectrum, and a HRMS (ESI) analysis of the compound represented by Structural Formula (1) are as follows.

¹H NMR (400 MHz, CD₃OD): δ 5.10 (d, J=3.8 Hz, 1H), 5.08 (d, J=3.9 Hz, 1H), 4.02 (dd, J=10.5 Hz, 2.7 Hz, 1H), 3.90 (t, J=9.4 Hz, 1H), 3.84 (br dt, J=9.4 Hz, 2.8 Hz, 1H), 3.80-3.76 (m, 5H), 3.67 (dd, J=11.7 Hz, 4.4 Hz, 1H), 3.66-3.63 (m, 1H), 3.54 (dd, J=7.8 Hz, 2.5 Hz, 1H), 3.49 (dd, J=9.6 Hz, 3.8 Hz, 1H), 3.46 (dd, J=9.6 Hz, 3.8 Hz, 1H), 3.31 (t, J=9.4 Hz, 1H), 3.28 (t, J=9.4 Hz, 1H), 1.19 (s, 3H), 1.17 (s, 3H).

¹³C NMR (100 MHz, CDCl₃): δ 95.1, 95.0, 80.4, 78.1, 74.7, 74.5, 74.4, 73.8, 73.4, 73.2, 72.8, 72.7, 71.9, 62.6, 62.1, 26.5, 25.4.

HRMS (ESI) analysis: calcd. for C₁₇H₃₂O₁₃Na m/z 467.1735 [M+Na]⁺, found 467.1725.

### Example 2

### Synthesis-1 of Lentztrehalose A Via Compound Represented by Structural Formula 4a′

<Synthesis of Compound Represented by Structural Formula 4a′ and Compound Represented by Structural Formula 4a>

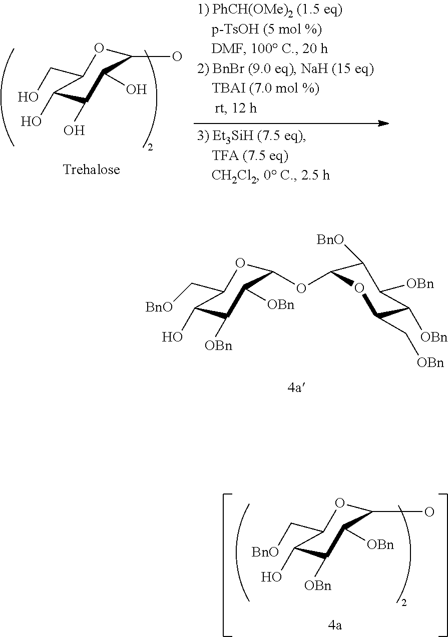

1) Trehalose (3.57 g, 10.4 mmol), benzaldehyde dimethylacetal (0.78 mL, 0.5 equivalents), and p-TsOH (44.8 mg, 2.5 mol %) were dissolved in DMF (50 mL) and then heated to 100° C. The reaction system was stirred under reduced pressure of 240 mmHg for 10 min. Then, benzaldehyde dimethylacetal (0.39 mL, 0.25 equivalents) was added thereto, and stirred under the same condition for 2 hours. Further, benzaldehyde dimethylacetal (0.39 mL, 0.25 equivalents) was added thereto, and continued to stir under reduced pressure of 240 mmHg for 2.5 hours. Further, benzaldehyde dimethylacetal (0.195 mL, 0.25 equivalents) was added thereto, and stirred under reduced pressure of 240 mmHg for 12 hours. Then, benzaldehyde dimethylacetal (0.195 mL, 0.25 equivalents) and p-TsOH (44.8 mg, 2.5 mol %) were further added thereto, and then stirred for up to 20 hours in total. The reaction system was cooled to room temperature and then neutralized with triethylamine. The resultant mixed solution was used in the next reaction without further purification.

2) To the mixed solution, were added sodium hydride (6.24 g, 15 equivalents), TBAI (268 mg, 7 mol %), and benzyl bromide (11.1 mL, 9 equivalents) sequentially under ice cooling, followed by stirring at room temperature for 12 hours. The resultant reaction liquid was poured into ice-water, extracted with methylene chloride, and concentrated to dryness. The residue was purified by silica gel column chromatography (hexane:ethyl acetate=8:1 to 5:1) to thereby obtain a benzyl protected product (10.0 g).

3) The benzyl protected product was dissolved in methylene chloride (250 mL), followed by adding at 0° C. triethylsilane (12.4 mL, 7.5 equivalents) and trifluoroacetic acid (5.97 mL, 7.5 equivalents) thereto and then stirring at the same temperature for 2.5 hours. The reaction system was diluted with methylene chloride, and washed with saturated aqueous sodium bicarbonate solution. Then, an organic layer was concentrated to dryness. The residue was purified by silica gel column chromatography (hexane:ethyl acetate=6:1 to 3:1) to thereby obtain the compound represented by Structural Formula 4a′ (4.00 g, 4.11 mmol, 39% in 3 steps) as colorless syrup and the compound represented by Structural Formula 4a (1.26 g, 1.43 mmol, 14% in 3 steps) as white powder.

Results of a specific rotation, a ¹H NMR spectrum, a ¹³C NMR spectrum, and a HRMS (ESI) analysis of the compound represented by Structural Formula 4a′ were the same as in Example 1.

Results of a melting point, a specific rotation, a ¹H NMR spectrum, a ¹³C NMR spectrum, and a HRMS (ESI) analysis of the compound represented by Structural Formula 4a were also the same as in Example 1.

<Synthesis of Compound Represented by Structural Formula 3a′>

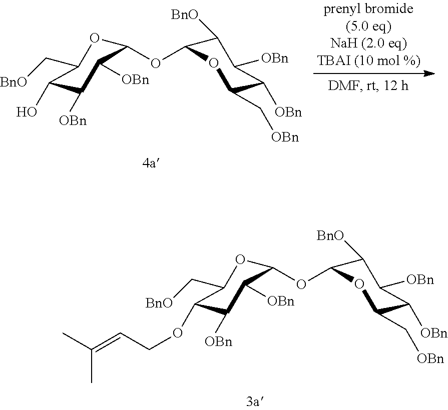

The compound represented by Structural Formula 4a′ (106 mg, 0.109 mmol) was dissolved in DMF (2 mL), followed by adding TBAI (2.5 mg, 10 mol %), sodium hydride (8.7 mg, 2 equivalents), and prenyl bromide (62.9 μL, 5 equivalents) thereto sequentially under ice cooling and then stirring at room temperature for 12 hours. Methanol (0.1 mL) was added thereto, and stirred for 5 min. Then, distilled water (10 mL) and methylene chloride (10 mL) were added thereto. An organic layer was separated, washed with aqueous hydrochloric acid solution (1 M, 5 mL) and saturated aqueous sodium bicarbonate solution (5 mL), and then dried over sodium sulfate. The solvent was distilled off, followed by purifying by silica gel column chromatography (hexane:ethyl acetate=5:1) to thereby obtain the compound represented by Structural Formula 3a′ (115 mg, 0.109 mmol) quantitatively.

—Compound Represented by Structural Formula 3a′—

Results of a specific rotation, a ¹H NMR spectrum, a ¹³C NMR spectrum, and a HRMS (ESI) analysis of the compound represented by Structural Formula 3a′ are as follows.

Specific rotation: [α]²⁵_{D}=87 (c=1.6, CHCl₃).

¹H NMR (400 MHz, CDCl₃): δ 7.38-7.20 (m, 33H), 7.12-7.09 (m, 2H), 5.26-5.24 (m, 1H), 5.21-5.20 (m, 2H), 4.99 (d, J=10.8 Hz, 1H), 4.97 (d, J=11.0 Hz, 1H), 4.86 (d, J=10.8 Hz, 1H), 4.84 (d, J=11.0 Hz, 1H), 4.80 (d, J=10.5 Hz, 1H), 4.70-4.62 (m, 4H), 4.56 (d, J=12.1 Hz, 1H), 4.54 (d, J=12.2 Hz, 1H), 4.44 (d, J=12.1 Hz, 1H), 4.43 (d, J=10.5 Hz, 1H), 4.35 (d, J=12.2 Hz, 1H), 4.33-4.27 (m, 1H), 4.17-4.13 (m, 2H), 3.99 (t, J=9.4 Hz, 2H), 3.96 (t, J=9.4 Hz, 1H), 3.69-3.65 (m, 1H), 3.58-3.47 (m, 5H), 3.41 (d, J=10.8 Hz, 1H), 3.33 (d, J=10.8 Hz, 1H), 1.69 (s, 3H), 1.53 (s, 3H).

¹³C NMR (100 MHz, CDCl₃): δ 139.1, 139.0, 138.4, 138.4, 138.3, 138.0, 138.0, 137.2, 128.5, 128.5, 128.4, 128.4, 128.1, 128.1, 128.0, 128.0, 127.8, 127.7, 127.6, 127.6, 127.6, 127.5, 121.3, 94.8, 82.0, 81.8, 79.4, 79.4, 77.7, 77.5, 77.4, 75.8, 75.7, 75.2, 73.6, 72.8, 72.7, 70.8, 70.6, 69.7, 68.4, 68.1, 25.9, 18.1.

HRMS (ESI) analysis: calcd. for C₆₆H₇₂O₁₁Na m/z 1063.4967 [M+Na]⁺, found 1063.4945.

<Synthesis of Compound Represented by Structural Formula 2a′>

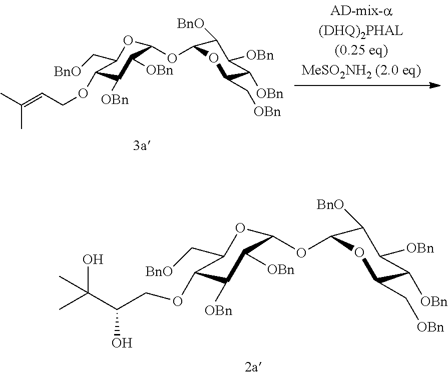

The compound represented by Structural Formula 3a′ (46.9 mg, 45 μmol) was dissolved in a mixed solvent of t-butanol, water, and acetone (1:1:1), followed by stirring with AD-mix-α (124 mg), (DHQ)₂PHAL (9.2 mg, 0.25 equivalents), and methane sulfonamide (8.9 mg, 2 equivalents) at 0° C. for 72 hours. The reaction system was neutralized by adding sodium sulfite thereto and stirring at 0° C. for 30 min, and then extracted with ethyl acetate. An organic layer was washed with saturated aqueous sodium chloride solution, dried over sodium sulfate, and then concentrated to dryness. The diastereomeric ratio was found to be 9:1 by ¹H NMR. The residue was purified by silica gel column chromatography (hexane:ethyl acetate=3:1) to thereby obtain the compound represented by Structural Formula 2a′ (21.9 mg, 20.4 μmol, 45%).

—Compound Represented by Structural Formula 2a′—

Results of a ¹H NMR spectrum, a ¹³C NMR spectrum, and a HRMS (ESI) analysis of the compound represented by Structural Formula 2a′ are as follows.

¹H NMR (400 MHz, CDCl₃): δ 7.38-7.13 (m, 33H), 7.12-7.09 (m, 2H), 5.20 (d, J=3.4 Hz, 1H), 5.19 (d, J=3.4 Hz, 1H), 5.02 (d, J=10.8 Hz, 1H), 5.01 (d, J=10.8 Hz, 1H), 4.89 (d, J=10.8 Hz, 1H), 4.81 (d, J=10.5 Hz, 1H), 4.77 (d, J=10.8 Hz, 1H), 4.72 (d, J=12.1 Hz, 1H), 4.68-4.61 (m, 2H), 4.64 (d, J=12.1 Hz, 1H), 4.54 (d, J=12.1 Hz, 1H), 4.53 (d, J=12.1 Hz, 1H), 4.45 (d, J=10.5 Hz, 1H), 4.40 (d, J=12.1 Hz, 1H), 4.37 (d, J=12.1 Hz, 1H), 4.15 (br dt, J=9.8 Hz, 3.4 Hz, 1H), 4.14-06 (m, 2H), 3.91 (t, J=9.4 Hz, 1H), 3.82 (dd, J=9.4 Hz, 2.3 Hz, 1H), 3.69 (t, J=9.4 Hz, 1H), 3.60 (dd, J=9.6 Hz, 3.4 Hz, 1H), 3.54 (dd, J=9.4 Hz, 3.6 Hz, 1H), 3.52 (dd, J=9.4 Hz, 3.6 Hz, 1H), 3.49 (dd, J=9.6 Hz, 8.0 Hz, 2H), 3.44-3.41 (m, 1H), 3.41-3.39 (m, 1H), 3.37 (dd, J=10.8 Hz, 1.8 Hz, 1H), 3.32 (dd, J=10.8 Hz, 2.3 Hz, 1H), 3.00 (d, J=3.9 Hz, 1H), 2.40 (s, 1H), 1.10 (s, 3H), 0.98 (s, 3H).

¹³C NMR (100 MHz, CDCl₃): δ 138.9, 138.6, 138.4, 138.3, 138.1, 137.9, 137.7, 128.5, 128.5, 128.5, 128.1, 128.1, 128.0, 127.9, 127.9, 127.8, 127.8, 127.8, 127.8, 127.7, 127.5, 127.5, 94.6, 94.4, 82.0, 81.3, 79.6, 79.6, 78.2, 77.8, 76.3, 75.7, 75.7, 75.3, 73.8, 73.7, 73.6, 73.0, 72.6, 71.3, 70.8, 70.5, 68.5, 68.2, 26.6, 24.6.

HRMS (ESI) analysis: calcd. for C₆₆H₇₄O₁₃Na m/z 1097.5022 [M+Na]⁺, found 1097.4999.

### <Synthesis of Compound Represented by Structural Formula (1)>

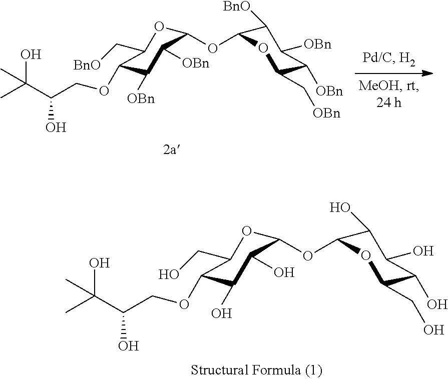

The compound represented by Structural Formula 2a′ (18.0 mg, 28.4 μmol) was dissolved in methanol (1.5 mL). To this, was added 10% Pd/C (15 mg), followed by stirring under a hydrogen atmosphere for 24 hours. After filtering the catalyst off through Celite, the resultant filtrate was concentrated to dryness to thereby obtain the compound represented by Structural Formula (1) (lentztrehalose A) (9.0 mg, 28.4 μmol) quantitatively (6 steps, yield: 18%).

Results of a ¹H NMR spectrum, a ¹³C NMR spectrum, and a HRMS (ESI) analysis of the compound represented by Structural Formula (1) were the same as in Example 1.

### Example 3

### Synthesis-2 of Lentztrehalose A Via Compound Represented by Structural Formula 4a′

<Synthesis of Compound Represented by Structural Formula 2a″>

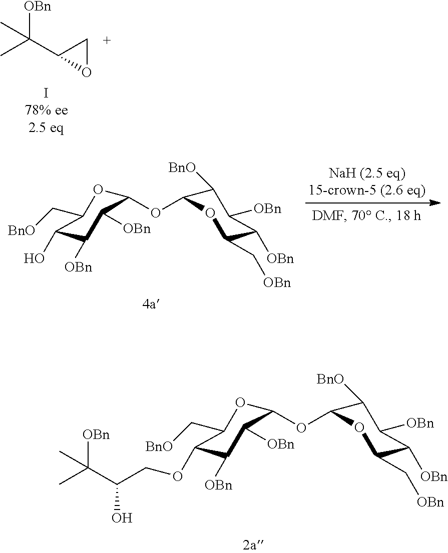

The compound represented by Structural Formula 4a′ was obtained in the same manner as in Example 2.

The compound represented by Structural Formula 4a′ (25.0 mg, 25.1 μmol) was dissolved in dimethylformamide (1.5 mL), followed by adding sodium hydride (2.6 mg, 2.5 equivalents), 15-crown-5 (13 μL, 2.6 equivalents), and epoxide represented by the Structural Formula I (12.3 mg, 2.5 equivalents) sequentially under ice cooling and then stirring at 70° C. for 18 hours. Methanol (0.1 mL) was added thereto under ice cooling, and stirred for 5 min. Then, distilled water (8 mL) and methylene chloride (10 mL) were added thereto. An organic layer was separated, washed with aqueous hydrochloric acid solution (1 N, 4 mL) and saturated aqueous sodium bicarbonate solution (5 mL), and dried over sodium sulfate. After work-up, the resultant was purified by silica gel column chromatography to thereby obtain the compound represented by Structural Formula 2a″ (9.0 mg, 7.72 μmol, yield: 30%).

—Compound Represented by Structural Formula 2a″—

Results of a ¹H NMR spectrum and a HRMS (ESI) analysis of the compound represented by Structural Formula 2a″ are as follows.

¹H NMR (400 MHz, CDCl₃): δ 7.41-7.24 (m, 36H), 7.14-7.11 (m, 2H), 5.21 (d, J=3.4 Hz, 2H), 5.02 (d, J=11.0 Hz, 1H), 5.00 (d, J=10.5 Hz, 1H), 4.88 (d, J=11.0 Hz, 1H), 4.87 (d, J=10.5 Hz, 1H), 4.82 (d, J=10.5 Hz, 1H), 4.71-4.64 (m, 4H), 4.55 (d, J=12.1 Hz, 1H), 4.49 (d, J=11.9 Hz, 1H), 4.47 (d, J=10.5 Hz, 1H), 4.44-4.42 (m, 2H), 4.42 (d, J=11.9 Hz, 1H), 4.37 (d, J=12.1 Hz, 1H), 4.18-4-4.06 (m, 2H), 4.03 (t, J=9.4 Hz, 1H), 4.00 (t, J=9.4 Hz, 1H), 3.97-3.93 (m, 1H), 3.85 (dd, J=10.3 Hz, 1.6 Hz, 1H), 3.75-3.63 (m, 3H), 3.58-3.46 (m, 5H), 3.42-3.34 (m, 2H), 1.15 (s, 3H), 1.11 (s, 3H).

HRMS (ESI) analysis: calcd. for C₇₃H₈₀O₁₃Na m/z 1187.5491 [M+Na]⁺, found 1187.5476.

### <Synthesis of Compound Represented by Structural Formula (1)>

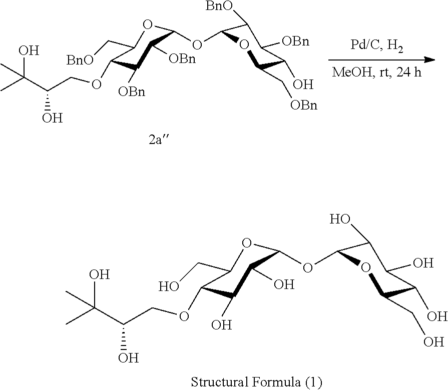

The compound represented by Structural Formula 2a″ (8.5 mg, 7.29 μmol) was dissolved in methanol (1 mL). To this, was added 10% Pd/C (7.0 mg), followed by stirring under a hydrogen atmosphere for 24 hours. After filtering the catalyst off through Celite, the resultant filtrate was concentrated to dryness to thereby obtain the compound represented by Structural Formula (1) (lentztrehalose A) (2.9 mg, 6.53 μmol, yield: 89%). Note that, absolute configuration of the lentztrehalose A, which had been synthesized utilizing a stereospecific ring-opening reaction between a synthetic intermediate derived from trehalose with known absolute configuration and epoxide with known absolute configuration, was determined as represented by the Structural Formula (1).

Results of a ¹H NMR spectrum, a ¹³C NMR spectrum, and a HRMS (ESI) analysis of the resultant compound represented by Structural Formula (1) were the same as in Example 1.

From the above results, it was demonstrated that the compound represented by Structural Formula (1) (lentztrehalose A) could be synthesized with high efficiency by the methods according to the present invention.

Aspects of the present invention are, for example, as follows.

<1> A method for producing a compound represented by the following Structural Formula (1), the method including:

introducing a benzyl group into trehalose to thereby produce at least one of a compound represented by the following Structural Formula 4a and a compound represented by the following Structural Formula 4a′;

subjecting at least one of the compound represented by the following Structural Formula 4a and the compound represented by the following Structural Formula 4a′ to prenylation to thereby produce at least one of a compound represented by the following Structural Formula 3a and a compound represented by the following Structural Formula 3a′;

subjecting at least one of the compound represented by the following Structural Formula 3a and the compound represented by the following Structural Formula 3a′ to sharpless asymmetric dihydroxylation to thereby produce at least one of a compound represented by the following Structural Formula 2a and a compound represented by the following Structural Formula 2a′; and

allowing at least one of the compound represented by the following Structural Formula 2a and the compound represented by the following Structural Formula 2a′ to react with hydrogen in the presence of a palladium catalyst to thereby produce the compound represented by the following Structural Formula (1):

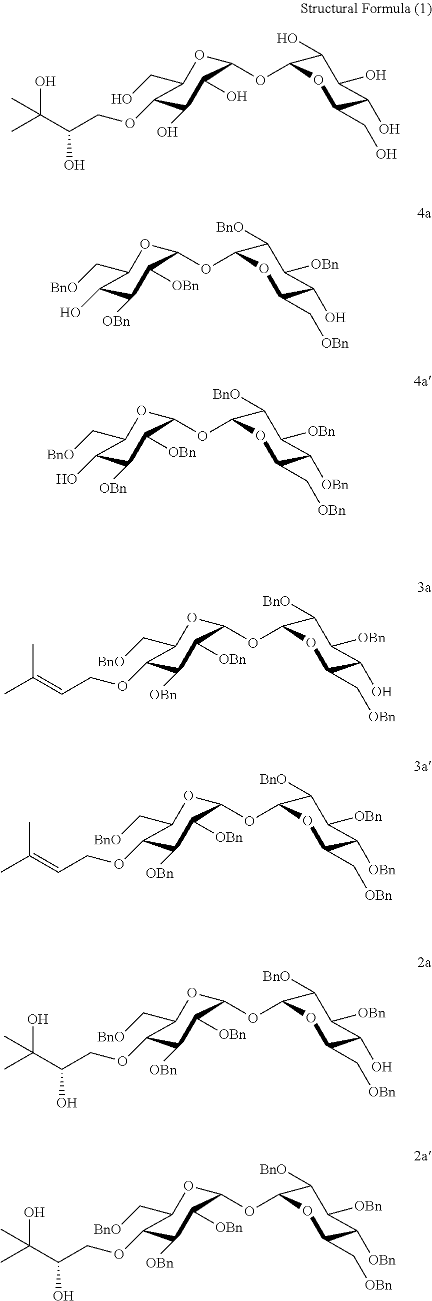

wherein Bn in the Structural Formula 4a, the Structural Formula 4a′, the Structural Formula 3a, the Structural Formula 3a′, the Structural Formula 2a, and the Structural Formula 2a′ denotes a benzyl group.

<2> A method for producing a compound represented by the following Structural Formula (1), the method including:

introducing a benzyl group into trehalose to thereby produce a compound represented by the following Structural Formula 4a′;

allowing the compound represented by the following Structural Formula 4a′ to react with epoxide represented by the following Structural Formula I to thereby produce a compound represented by the following Structural Formula 2a″; and

allowing the compound represented by the following Structural Formula 2a″ to react with hydrogen in the presence of a palladium catalyst to thereby produce the compound represented by the following Structural Formula (1):

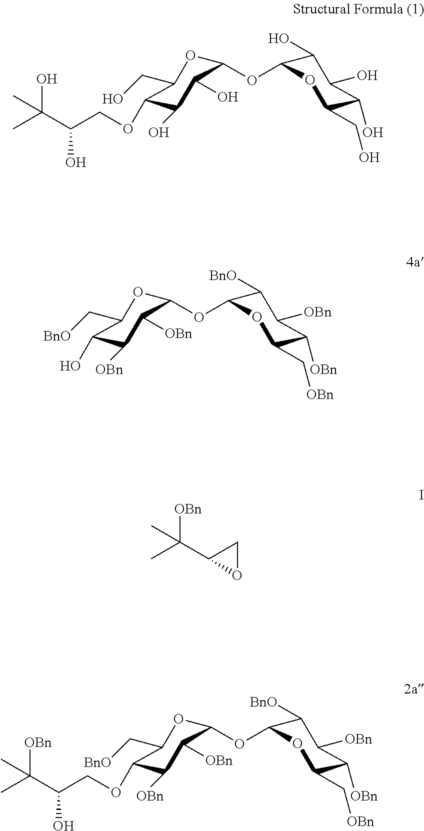

wherein Bn in the Structural Formula 4a′, the Structural Formula I, and the Structural Formula 2a″ denotes a benzyl group.

<3> A method for producing a compound represented by the following Structural Formula (1), the method including:

allowing at least one of a compound represented by the following Structural Formula 2a, a compound represented by the following Structural Formula 2a′, and a compound represented by the following Structural Formula 2a″ to react with hydrogen in the presence of a palladium catalyst:

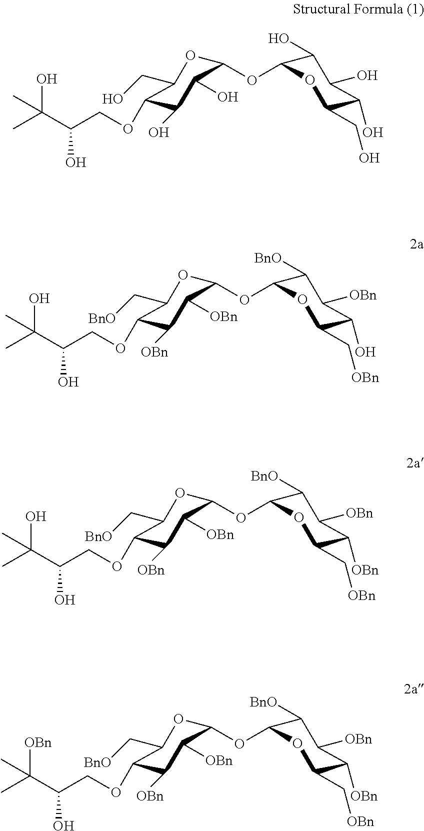

wherein Bn in the Structural Formula 2a, the Structural Formula 2a′, and the Structural Formula 2a″ denotes a benzyl group.

<4> A method for producing at least one of a compound represented by the following Structural Formula 2a and a compound represented by the following Structural Formula 2a′, the method including:

subjecting at least one of a compound represented by the following Structural Formula 3a and a compound represented by the following Structural Formula 3a′ to sharpless asymmetric dihydroxylation:

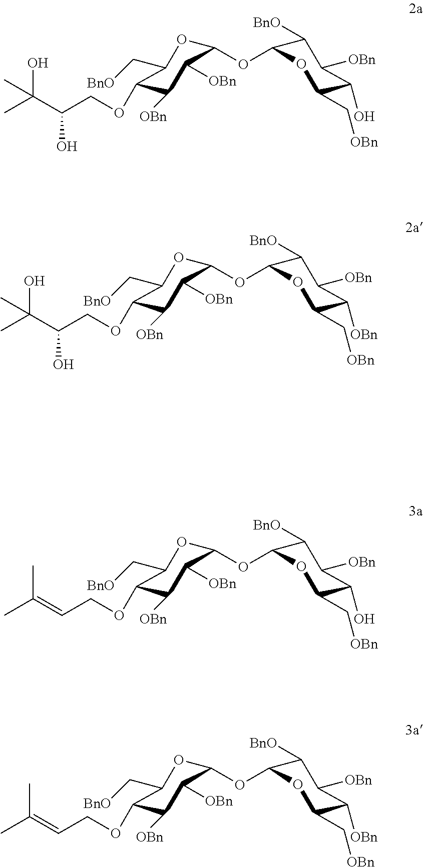

wherein Bn in the Structural Formula 2a, the Structural Formula 2a′, the Structural Formula 3a, and the Structural Formula 3a′ denotes a benzyl group.

<5> A method for producing a compound represented by the following Structural Formula 2a″, the method including:

allowing a compound represented by the following Structural Formula 4a′ to react with epoxide represented by the following Structural Formula I:

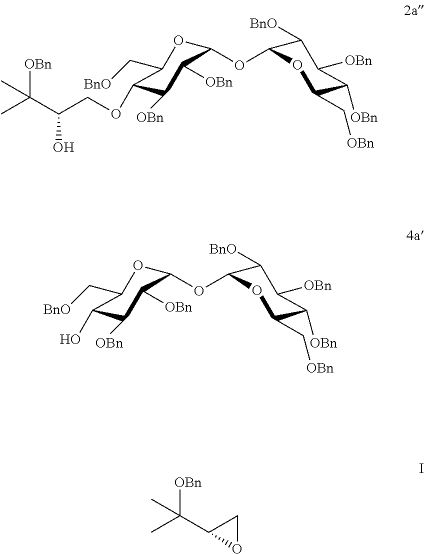

wherein Bn in the Structural Formula 2a″, the Structural Formula 4a′, and the Structural Formula I denotes a benzyl group.

<6> A method for producing a compound represented by the following Structural Formula 3a or the following Structural Formula 3a′, the method including:

subjecting at least one of a compound represented by the following Structural Formula 4a and a compound represented by the following Structural Formula 4a′ to prenylation:

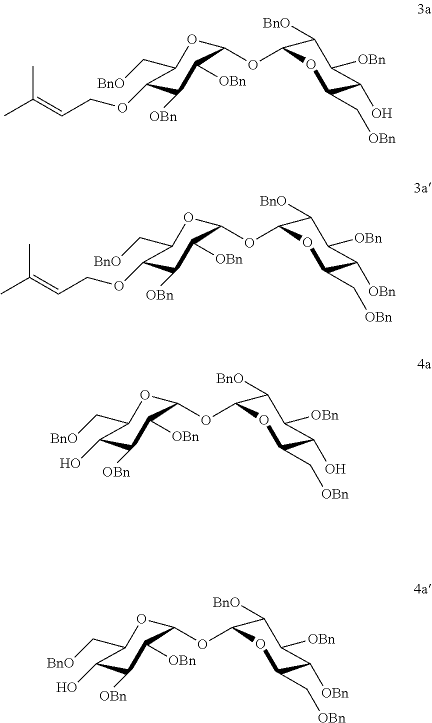

wherein Bn in the Structural Formula 3a, the Structural Formula 3a′, the Structural Formula 4a, and the Structural Formula 4a′ denotes a benzyl group.

<7> A method for producing a compound represented by the following Structural Formula 4a or the following Structural Formula 4a′, the method including:

introducing a benzyl group into trehalose:

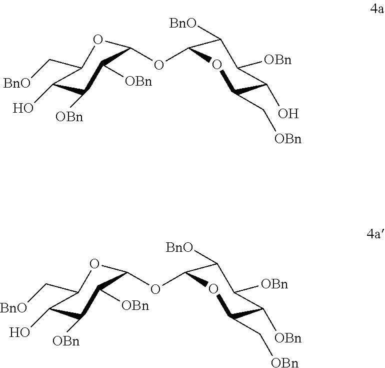

wherein Bn in the Structural Formula 4a and the Structural Formula 4a′ denotes a benzyl group.

<8> A compound represented by the following Structural Formula 2a, the following Structural Formula 2a′, or the following Structural Formula 2a″:

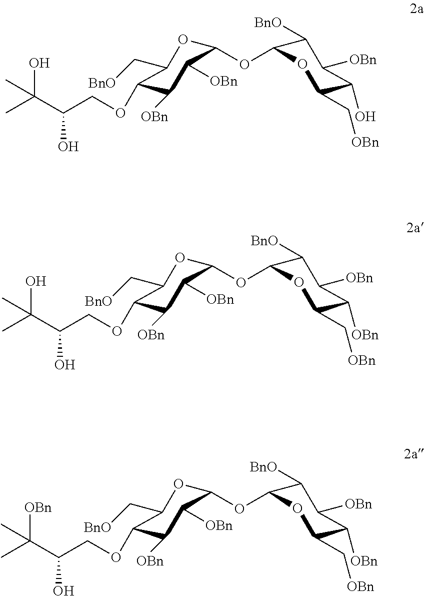

wherein Bn in the Structural Formula 2a, the Structural Formula 2a′, and the Structural Formula 2a″ denotes a benzyl group.

<9> A compound represented by the following Structural Formula 3a or the following Structural Formula 3a′:

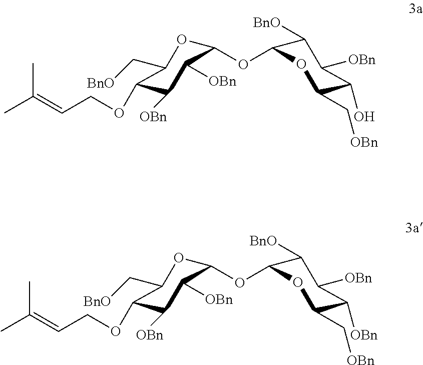

wherein Bn in the Structural Formula 3a and the Structural Formula 3a′ denotes a benzyl group.

<10> A compound represented by the following Structural Formula 4a or the following Structural Formula 4a′:

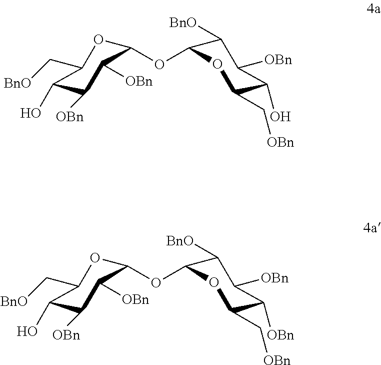

wherein Bn in the Structural Formula 4a and the Structural Formula 4a′ denotes a benzyl group.

What is claimed is:

1. A method for producing a compound represented by the following Structural Formula (1), the method comprising:
introducing a benzyl group into trehalose to thereby produce at least one of a compound represented by the following Structural Formula 4a and a compound represented by the following Structural Formula 4a′;
subjecting at least one of the compound represented by the following Structural Formula 4a and the compound represented by the following Structural Formula 4a′ to prenylation to thereby produce at least one of a compound represented by the following Structural Formula 3a and a compound represented by the following Structural Formula 3a′;
subjecting at least one of the compound represented by the following Structural Formula 3a and the compound represented by the following Structural Formula 3a′ to sharpless asymmetric dihydroxylation to thereby produce at least one of a compound represented by the following Structural Formula 2a and a compound represented by the following Structural Formula 2a′; and
allowing at least one of the compound represented by the following Structural Formula 2a and the compound represented by the following Structural Formula 2a′ to react with hydrogen in the presence of a palladium catalyst to thereby produce the compound represented by the following Structural Formula (1): 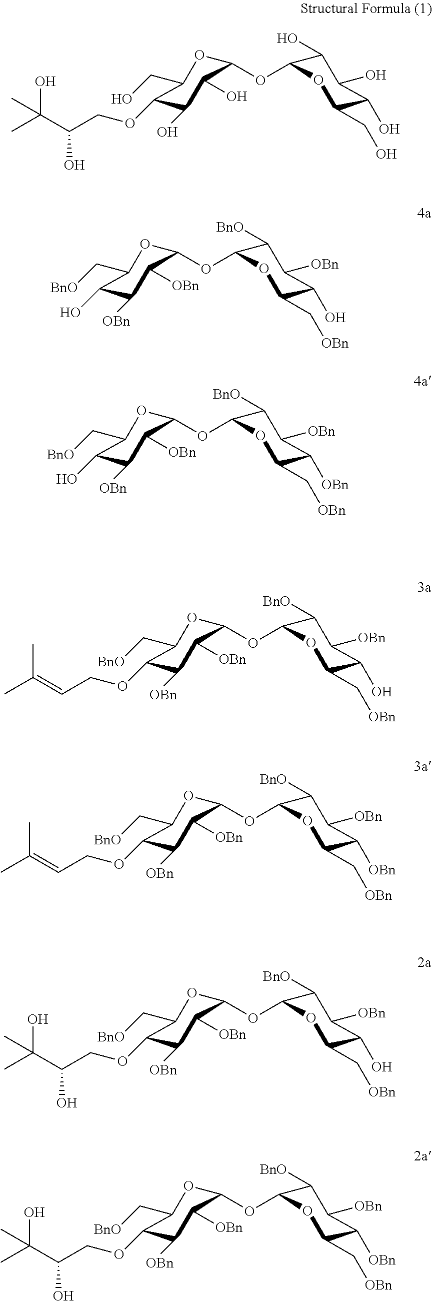
wherein Bn in the Structural Formula 4a, the Structural Formula 4a′, the Structural Formula 3a, the Structural Formula 3a′, the Structural Formula 2a, and the Structural Formula 2a′ denotes a benzyl group.

2. A method for producing a compound represented by the following Structural Formula (1), the method comprising:
introducing a benzyl group into trehalose to thereby produce a compound represented by the following Structural Formula 4a′;
allowing the compound represented by the following Structural Formula 4a′ to react with epoxide represented by the following Structural Formula I to thereby produce a compound represented by the following Structural Formula 2a″; and
allowing the compound represented by the following Structural Formula 2a″ to react with hydrogen in the presence of a palladium catalyst to thereby produce the compound represented by the following Structural Formula (1): 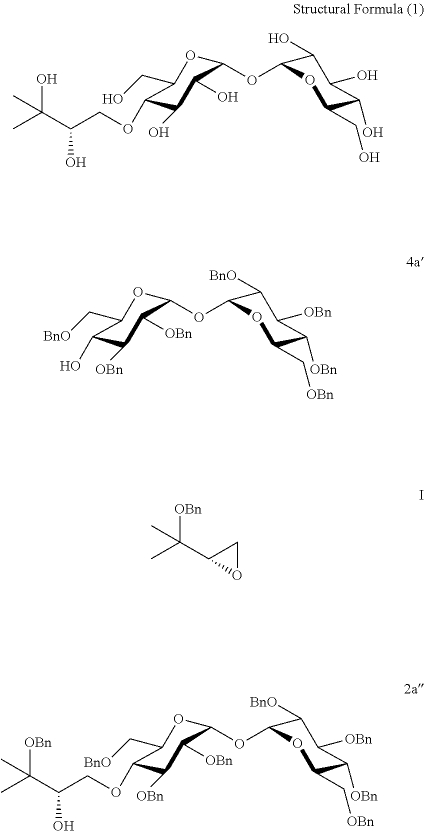
wherein Bn in the Structural Formula 4a′, the Structural Formula I, and the Structural Formula 2a″ denotes a benzyl group.

3. A method for producing a compound represented by the following Structural Formula (1), the method comprising:
allowing at least one of a compound represented by the following Structural Formula 2a, a compound represented by the following Structural Formula 2a′, and a compound represented by the following Structural Formula 2a″ to react with hydrogen in the presence of a palladium catalyst: 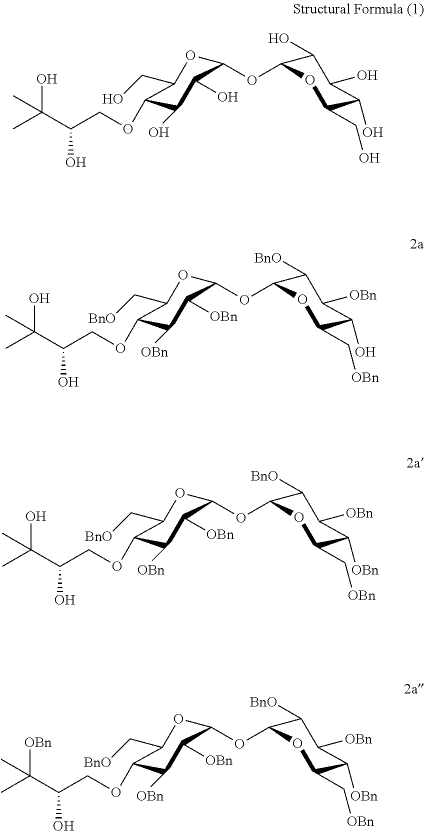
wherein Bn in the Structural Formula 2a, the Structural Formula 2a′, and the Structural Formula 2a″ denotes a benzyl group.

4. A method for producing a compound represented by the following Structural Formula 4a or the following Structural Formula 4a′, the method comprising:
introducing a benzyl group into trehalose: 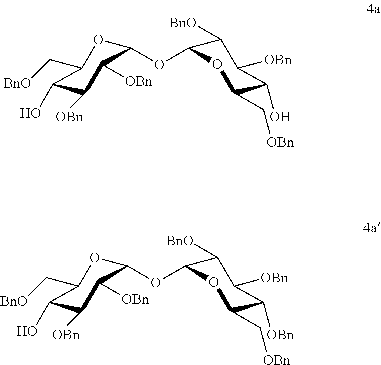
wherein Bn in the Structural Formula 4a and the Structural Formula 4a′ denotes a benzyl group.

5. A compound represented by the following Structural Formula 4a or the following Structural Formula 4a′: 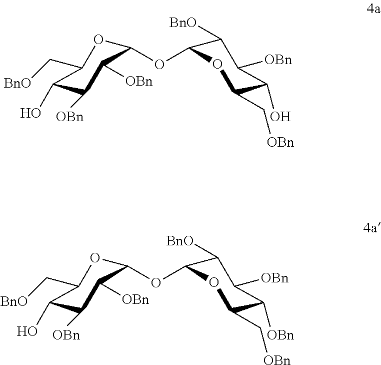
wherein Bn in the Structural Formula 4a and the Structural Formula 4a′ denotes a benzyl group.
